(19) United States
(12) Patent Application Publication
(10) Pub. No.: US 2011/0045462 A1
(43) Pub. Date: Feb. 24, 2011

(54) DIGITAL ANALYSIS OF GENE EXPRESSION

(75) Inventors: Xiang-Dong Fu, San Diego, CA (US); Michael G. Rosenfeld, San Diego, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/514,952

(22) PCT Filed: Nov. 14, 2007
(86) PCT No.: PCT/US07/24021

§ 371 Date: Nov. 9, 2010

Related U.S. Application Data
(60) Provisional application No. 60/865,724, filed on Nov. 14, 2006.

(51) Int. Cl. C12Q 1/68

(52) U.S. Cl. 435/6

(57) ABSTRACT

The disclosure provides methods and compositions useful for high throughput sequencing of nucleic acid sequences associated with gene expression, nucleic acid-polypeptide interactions, and/or chromosomal interactions.

### CROSS REFERENCE TO RELATED APPLICATIONS

This application claims priority under 35 U.S.C. §119 from Provisional Application Ser. No. 60/865,724, filed Nov. 14, 2006, the disclosure of which is incorporated herein by reference.

### TECHNICAL FIELD

Provided herein are methods and compositions useful for high throughput sequencing of nucleic acid sequences associated with gene expression, nucleic acid-polypeptide interactions, and/or chromosomal interactions.

### BACKGROUND

The elucidation of genomes for humans and other model organisms has made it possible to conduct analysis of gene expression and regulation at the genome scale. In post-genome cancer research new technologies are required to investigate the role of gene expression, nucleic acid-polypeptide interactions and chromosomal interactions in cell proliferation disorders.

### SUMMARY

The disclosure provide methods that allow detection of gene expression in a digital format and genome wide without RNA isolation, signal amplification, and array hybridization. The technology can be used to identify biomarkers in different cell types, responsive to special signals, associated with specific pathological conditions, including cancer at different stages.

Provided herein are methods of detection of gene expression in digital format and genome-wide without RNA isolation, signal amplification, and array hybridization. By coupling DASL (DNA Annealing, Selection, Ligation) with massive sequencing, it is possible to count the number of molecules from individual profiling experiments to obtain quantum information on gene expression. Quantitative information on gene expression can be obtained at defined cellular stages; therefore, the technology can be used to identify biomarkers for cancers and other diseases.

The methods of the disclosure overcome the defects associated with previous approaches and allow research to obtain quantitative information on gene expression that is directly proportional to specific mRNA copy numbers in the cell under investigation. This detection can be at the single cell level.

In one embodiment, a method for determining the sequence of a target polynucleotide is provided. The method includes: a) providing a nucleic acid template comprising a target polynucleotide, wherein the template comprises a capture moiety; b) generating a first hybridization complex by annealing to the target polynucleotide a primer pair comprising: i) a first oligonucleotide comprising a first portion comprising a target-specific terminal annealing domain partially or completely complementary to a first segment of the target polynucleotide, and a second portion comprising a universal landing site that is complementary to an amplification primer but not complementary to the target polynucleotide; ii) a second oligonucleotide comprising a first portion comprising a target-specific terminal annealing domain partially or completely complementary to a second segment of the target polynucleotide, and a second portion comprising a universal landing site that is complementary to an amplification primer but not complementary to the target polynucleotide, wherein the universal landing sites are not the same, and wherein the first oligonucleotide target-specific terminal annealing domain and second oligonucleotide target-specific terminal annealing domain generate a ligatable junction; c) ligating the ligatable junction to form a ligated probe; d) separating the ligated probe from the template; e) hybridizing the ligated probe to a first capture primer comprising a terminal end detachably linked to a solid substrate, and extending the first capture primer to form a first extended polynucleotide complementary to the ligated probe; f) removing the ligated probe and annealing the unlinked terminal end of the first extended polynucleotide to a second capture primer comprising a terminal end detachably linked to a solid substrate, and extending the second capture primer to form a second extended polynucleotide complementary to the first extended polynucleotide; g) optionally repeating part f), wherein a colony of polynucleotides detachably linked to a solid substrate and suitable for sequencing are formed; and h) determining the sequence of the target polynucleotide.

In some aspects, the first oligonucleotide or second oligonucleotide, or first oligonucleotide and second oligonucleotide, further comprise a zip code sequence. The zip code sequence may be juxtaposed between the terminal annealing domain and terminal amplification domain.

In some aspects the nucleic acid template is deoxyribonucleic acid (DNA), ribonucleic acid (RNA), or cDNA.

In other aspects, the target polynucleotide comprises a splice junction.

In another aspect the universal landing sites are selected from a T3 and T7 priming site. In another aspect, the capture moiety is biotin.

In yet another aspect, the first oligonucleotide or second oligonucleotide, or first oligonucleotide and second oligonucleotide comprises a detectable label. The detectable label is selected from the group consisting of an isotopic label; a magnetic, electrical, or thermal label; an enzymatic label; and a fluorescent or luminescent label.

In another embodiment, a method for determining the sequence of a target polynucleotide is provided. The method includes, a) providing a nucleic acid template comprising a target polynucleotide; b) annealing a first oligonucleotide to the template, wherein the first oligonucleotide comprises: i) a first portion comprising a random sequence that partially or completely hybridizes to the target polynucleotide; ii) a second portion comprising a universal landing site that is complementary to an amplification primer but not complementary to the target polynucleotide; and iii) a capture moiety, c) extending the first oligonucleotide in a template directed reaction and isolating a first random priming product; d) annealing a second oligonucleotide to the first random priming product, wherein the second oligonucleotide comprises: i) a first portion comprising a random sequence that partially or completely hybridizes to the first random priming product; and ii) a second portion comprising a universal landing site that is complementary to an amplification primer but not complementary to the first random priming product; e) extending the second oligonucleotide primer in a template directed reaction and generating a second random priming product annealed to the first random priming product thereby forming a product complex; f) binding the capture moiety associated with the product complex with a binding partner associated with a solid substrate; g) separating the second random priming product from the first random priming product; h) hybridizing the second random priming product to a first capture primer comprising a terminal end detachably linked to a solid substrate, and extending the first capture primer to form a first extended polynucleotide complementary to the ligated probe; i) removing the ligated probe and annealing the unlinked terminal end of the first extended polynucleotide to a second capture primer comprising a terminal end detachably linked to a solid substrate, and extending the second capture primer to form a second extended polynucleotide complementary to the first extended polynucleotide; j) optionally repeating part i), wherein a colony of polynucleotides detachably linked to a solid substrate and suitable for sequencing are formed; and k) determining the sequence of the target polynucleotide.

In some aspects, the universal primer comprises an oligo-dT domain for hybridizing to mRNA.

In some aspects, the template nucleic acid is obtained from a chromatin immunoprecipitation (ChIP) assay. In other aspects, the template nucleic acid is obtained from a chromosome conformation capture (3C) assay.

The method of the disclosure comprises in situ hybridization of an oligonucleotide pool comprising at least two adjacent oligonucleotides to a target polynucleotide, in situ ligation of hybridized probes and high throughput sequencing of the ligated products.

The disclosure provides a method comprising contacting a tissue or biological sample with a pair of oligonucleotides, each oligonucleotide comprising two domains, a first domain comprising a sequence complementary to a target polynucleotide and a second domain comprising a universal primer. The oligonucleotide pairs are designed to be adjacent once hybridized. By adjacent is meant sufficiently close to allow proper ligation in the presence or absence of nucleotides. Contacting of the tissue or biological sample is performed under in situ hybridization conditions. Non-hybridized oligonucleotides are removed and hybridized oligonucleotides are ligated together with a ligase under in situ ligation conditions. The ligated oligonucleotides are eluted and subject to sequencing. In one aspect, specific subsamples (e.g., subsection and individual cells) are isolated by laser capture techniques followed by elution of the ligated oligonucleotides.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-C depicts the DASL scheme for gene expression profiling. (A) the DASL scheme, (B) technical repeats and (C) biological repeats.

FIG. 2 depicts RASL/DASL approaches modified to incorporate features for high throughput sequencing.

FIG. 3 depicts a molecular barcode strategy for multiplex sequencing.

FIG. 4 depicts coupling DASL with chromatin immunoprecipitation (ChIP).

FIG. 5 depicts chromosome conformation capture (3C) with DNA selection ligation. The technique may be referred to as 3D by coupling the standard chromosomal conformation capture (3C) assay with DNA Selection and Ligation (DSL).

FIG. 6 depicts a strategy to couple DASL with laser capture to profile gene expression in tumors.

FIG. 7 depicts oligonucleotide pairs for each target gene in which the 5′ oligo is flanked with a universal sequencing primer (A) and the 3′ oligo is flanked with another universal sequence (e.g. T7 primer).

FIGS. 8A and B show (A) a single molecule amplification on a sequencing slide. (B) Conversion of a DASL product to the configuration for sequencing by two rounds of primer extension. Note that both fusion primers contain 3 base pair differences in the middle compared to the ends of each DASL product to prevent primer extension from the DASL products.

FIG. 9 depicts an exemplary random priming-based technology.

FIG. 10 depicts exemplary methods provided herein for random priming-based sequencing technology in profiling gene expression.

FIG. 11 depicts the results of a series of experiments using double random priming methods described herein.

FIG. 12 depicts two exemplary strategies to map DNA-DNA interactions.

FIG. 13 depicts a hypothetic gene fusion event.

### DETAILED DESCRIPTION

As used herein and in the appended claims, the singular forms “a,” “and,” and “the” include plural referents unless the context clearly dictates otherwise. Thus, for example, reference to “a probe” includes a plurality of such cells and reference to “the primer” includes reference to one or more primers and equivalents thereof known to those skilled in the art, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure.

Recent development of massive sequencing technologies offers a promising solution to analyze RNA that is insufficient for microarray analysis. Two such sequencing platforms are now commercially available, one from 454 Inc. and the other from Solexa. Both platforms utilize a similar strategy to amplify single DNA molecule tethered either to beads (454 Inc.) or to a solid surface (Solexa) coated with a capture oligonucleotide probe. Both platforms require conversion of dsDNA into ssDNA, each molecule of which is flanked by two unique universal primers. While both platforms can generate a huge number of sequencing reads (˜0.4 million on the 454 platform and ten to hundreds of million on the Solexa platform) in a signal run. It is striking to note that the RASL/DASL products (ligated oligonucleotide pairs) are exactly configured for the massive sequencing strategy on either platform. By coupling DASL with massive sequencing, then, it may be possible to count the number of molecules from individual profiling experiments to obtain quantum information on gene expression.

The 454 platform uses capture of single DNA molecule per bead for subsequent emulsion PCR, which requires input DNA to be carefully titrated before loading onto the beads. In contrast, the Solexa platform uses a primer-coated surface to capture input DNA for local PCR amplification similar to the Polony assay, the technology that can accommodate a wide range of initial DNA concentration and thus has the capacity to produce hundreds of million sequences per run. The disclosure provides a sequencing-based gene expression profiling technology by coupling RASL/DASL assays with sequencing on a 454 Inc. or Solexa platform.

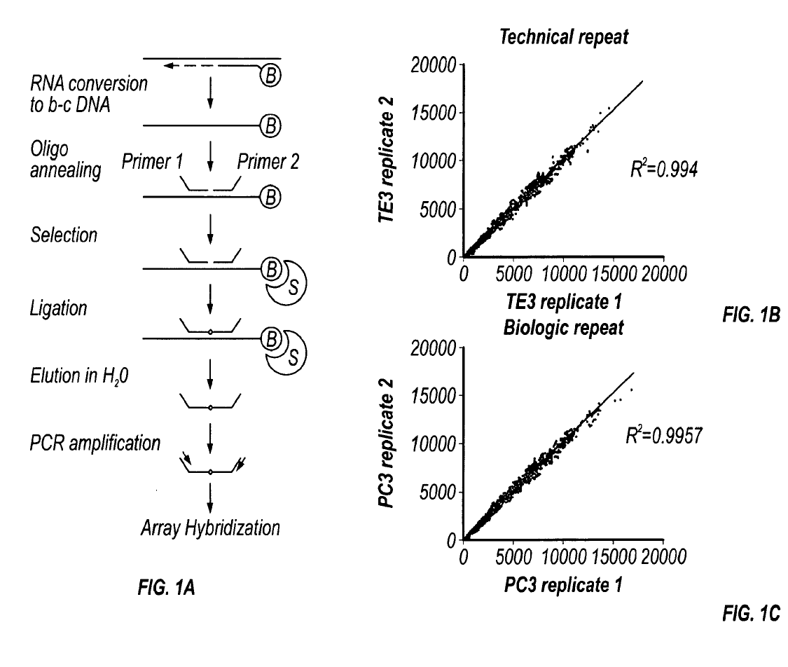

The essence of the RASL/DASL technology is to use RNA/DNA to template oligonucleotide ligation. As diagrammed in FIG. 1, isolated total RNA can be directly labeled in a photoactive biotinylation reaction or first converted to cDNA using biotinylated random primers. A pair of oligonucleotides, each of which is flanked by a universal primer, is used to target a unique 40mer sequence in a gene (note that strand sense of the oligonucleotides will depend on the choice of RNA or cDNA as template; here cDNA is used to illustrate the approach). Multiple pairs of oligonucleotides are pooled for multiplex analysis. The oligonucleotide pool is annealed to biotinylated cDNA, which can be driven to completion by using an excessive amount of oligonucleotides. After the annealing reaction, the annealed oligonucleotides are selected by streptavidin and free oligonucleotides are washed away. This selection step minimizes random oligonucleotide ligation by collision in solution containing a high concentration of oligonucleotides. A ligase (T4 kinase for RNA templated oligonucleotide ligation, Taq ligase for DNA templated oligonucleotide ligation) is next used to link the oligonucleotide pair that is aligned on DNA template side by side without any gap. This step selects oligonucleotides specifically annealed to intended target sequences because the chance for ligation of two unrelated, relatively immobilized oligonucleotides due to nonspecific sticking is in theory negligible. The ligation reaction converts two half-amplicoms to a full amplicom, which can be amplified by PCR using a pair of universal primers. The PCR products can be directly applied to a microarray for quantification.

The RASL/DASL scheme provides both specificity and sensitivity. The specificity is related to hybridization and ligation of correctly targeted oligonucleotides, instead of hybridization alone, ligated signals can be amplified for subsequent detection on microarray. The sensitivity is significantly elevated by PCR amplification of ligated oligonucleotides, all of which carry the same PCR landing sites in both ends and are uniform in length. Besides the improvement in specificity and sensitivity, the RASL/DASL assay permits analysis of extensively fragmented RNA, such as those from FFPE sections, so long as fragmented RNA has sufficient length to template oligonucleotide annealing and ligation. As shown in FIGS. 1B and 1C, both technical and biological repeats of the DASL analysis exhibit remarkable consistency.

The DASL technology can be used in an addressable approach. In this embodiment, one of the oligonucleotides in each pair is linked to a unique molecular barcode (a unique 20mer sequence that does not have significant sequence homology to any sequences in the human genome). This allows hybridization of PCR products to a universal zipcode array. This approach has been used to profile signature mRNA isoforms using 50 to 100 ng total RNA from various prostate cancer cell lines as well as prostate cancer FFPE tissues.

To overcome the limitation in zipcode choice, the DASL approach has been used to conduct genome-wide promoter array analysis by chromatin immunoprecipitation (ChIP). In this application, a unique 40mer in the promoter proximal region in each gene in the human genome was identified and used to fabricate a 40mer promoter array. Corresponding to each 40mer, a pair of assay oligonucleotides, each of which consists of a 20 nt sequence for targeting and a universal primer landing site for PCR were developed. Using this approach a pool containing ˜20,000 pair of oligonucleotides can work together in a single reaction to identify specific promoters occupied in vivo by both general and sequence specific DNA binding transcription factors. This represents the highest multiplex assay ever tested and the results have been extensively validated by quantitative PCR. The data show that targeting one region per gene is sufficient to obtain high quality results because the specificity ensured by ligation. The disclosure extends this RASL/DASL assay to genome-wide profiling experiments. The disclosure provides a system (without using the zipcode array) to target individual annotated transcripts in the human genome for gene expression profiling.

The disclosure provides a systematic approach to sequencing-based methods using DASL. For example, in one aspect, the disclosure develops the technology by designing an oligonucleotide set to target all annotated transcripts in the human genome. As and example, breast cancer FFPE sections can be used to develop the oligonucleotide set. A microarray-based approach will be used for gene expression profiling to obtain data that can be used for comparison with those from the sequencing-based approach.

Both Illumina and Agilent have developed microarrays for gene expression profiling using one or a few unique 50mer to target individual transcripts in the human genome. The RASL approach has been proven using relative intact RNA, which can be selected by biotinylated oligonucleotide-dT, while the DASL approach has been fully established with both intact and heavily fragmented RNA.

The sequence for each oligonucleotide probe on the Illumina array is available in the company's website, which contains ˜48,000 features, in which many genes are targeted by multiple probes. The methods of the disclosure can be used on annotated or non-annotated genes/sequences. For example, using annotated genes one probe per gene is selected, which will reduce the total number of probes to ˜18,000 (i.e., using the Illumina array) according to initial estimate. In addition, about 100 genes will be selected to prepare multiple oligonucleotides corresponding to all probes on the array, which report of the quality of individual experiments performed either on the array or by using the sequencing-approached described herein. Corresponding to each oligonucleotide probe sequence, the most unique 40mer region will be selected for the oligonucleotide design. An algorithm will be used for automatic oligonucleotide design, which prefers the maximal sequence uniqueness in the middle of each selected 40mer when blasting against the human genomic sequence.

Corresponding to each 40mer, two oligonucleotides are designed, each of which covers one half of the 40mer sequence and each linked to a universal primer landing site as diagrammed in FIG. 1A. One of the oligonucleotides in each pair will carry a 5′ phosphate, which will be incorporated during oligonucleotide synthesis. The quality of each oligonucleotide will be monitored by mass spectroscopy. In one aspect, all synthesized oligonucleotides will be pooled and adjusted to the final concentration of 0.1 pmole per oligonucleotide per assay in 10 ul.

Oligonucleotides are used in in situ hybridization techniques as further discussed herein. Tissue samples are prepared according to methods known in the art. For example, a biological sample and/or sub-sample comprises biological materials obtained from or derived from a living organism. Typically a biological sample will comprise proteins, polynucleotides, organic material, cells, tissue, and any combination of the foregoing. Such samples include, but are not limited to, hair, skin, tissue, cultured cells, cultured cell media, and biological fluids. A tissue is a mass of connected cells and/or extracellular matrix material (e.g., CNS tissue, neural tissue, eye tissue, placental tissue, mammary gland tissue, gastrointestinal tissue, musculoskeletal tissue, genitourinary tissue, and the like) derived from, for example, a human or other mammal and includes the connecting material and the liquid material in association with the cells and/or tissues. A biological fluid is a liquid material derived from, for example, a human or other mammal. Such biological fluids include, but are not limited to, blood, plasma, serum, serum derivatives, bile, phlegm, saliva, sweat, amniotic fluid, mammary fluid, and cerebrospinal fluid (CSF), such as lumbar or ventricular CSF. A sample also may be media containing cells or biological material.

In one aspect of the disclosure, a biological sample may be divided into two or more additional samples (e.g., subsamples). Typically, in such an instance, the biological sample is a tissue, such as a tissue biopsy. The systems and methods disclosed herein is also capable of analyzing tissue microarrays (e.g., a plurality of tissue samples on a single slide).

Typically, an individual sample used to prepare a subsample is embedded in embedding media such as paraffin or other waxes, gelatin, agar, polyethylene glycols, polyvinyl alcohol, celloidin, nitrocelluloses, methyl and butyl methacrylate resins or epoxy resins, which are polymerized after they infiltrate the specimen. Water soluble embedding media such as polyvinyl alcohol, carbowax (polyethylene glycols), gelatin, and agar, may be used directly on specimens. Water-insoluble embedding media such as paraffin and nitrocellulose require that specimens be dehydrated in several changes of solvent such as ethyl alcohol, acetone, or isopropyl alcohol and then be immersed in a solvent in which the embedding medium is soluble. In the case where the embedding medium is paraffin, suitable solvents for the paraffin are xylene, toluene, benzene, petroleum, ether, chloroform, carbon tetrachloride, carbon bisulfide, and cedar oil. Typically a tissue sample is immersed in two or three baths of the paraffin solvent after the tissue is dehydrated and before the tissue sample is embedded in paraffin. Embedding medium includes, for examples, any synthetic or natural matrix suitable for embedding a sample in preparation for tissue sectioning.

A tissue sample may be a conventionally fixed tissue sample, tissue samples fixed in special fixatives, or may be an unfixed sample (e.g., freeze-dried tissue samples). If a tissue sample is freeze-dried, it should be snap-frozen. Fixation of a tissue sample can be accomplished by cutting the tissue specimens to a thickness that is easily penetrated by fixing fluid. Examples of fixing fluids are aldehyde fixatives such as formaldehyde, formalin or formol, glyoxal, glutaraldehyde, hydroxyadipaldehyde, crotonaldehyde, methacrolein, acetaldehyde, pyruic aldehyde, malonaldehyde, malialdehyde, and succinaldehyde; chloral hydrate; diethylpyrocarbonate; alcohols such as methanol and ethanol; acetone; lead fixatives such as basic lead acetates and lead citrate; mercuric salts such as mercuric chloride; formaldehyde sublimates; sublimate dichromate fluids; chromates and chromic acid; and picric acid. Heat may also be used to fix tissue specimens by boiling the specimens in physiologic sodium chloride solution or distilled water for two to three minutes. Whichever fixation method is ultimately employed, the cellular structures of the tissue sample must be sufficiently hardened before they are embedded in a medium such as paraffin.

Using techniques such as those disclosed herein, a biological sample or a plurality of samples (e.g., from different subjects) comprising a tissue may be embedded, sectioned, and fixed.

According to experience on extracting RNA from FFPE sections, each 6 um FFPE section gives rise to total RNA in the range of 20 to 100 ng, the yield will depend on the size of each section and the cellular content in it. Various kits are commercially available to extract RNA from sections. Publicly available tissue sections are available. For example, FFPE breast cancer sections from the Moore Cancer Center of UCSD can be used as standards or to analyze gene expression. Processing of the tissue sections will comprise (1) RNA extraction from single FFPE sections, (2) quantification of total RNA yield in a nanodrop spectrometer, (3) determination of the degree in RNA fragmentation using, for example, the Aligent RNA analyzer, and (4) suspension of isolated RNA in a uniform concentration (e.g., 5 ng per 1 ul).

The range and quality of RNA from sections can be determined by processing multiple sections. In one aspect, samples that result in at least 20 ng total RNA with an average of length above 200 nt will be used in the methods of the disclosure. Isolated RNA can be directly biotinylated using a kit from Vector. In parallel, another aliquot from each sample is processed to convert RNA to biotinylated cDNA (b-cDNA) by random priming. This step will be accomplished by using the kit from Illumina, which is specifically designed to convert total RNA to b-cDNA for DASL analysis.

In order to ensure success in assaying the large number of cancer samples, the amount of RNA will be titrated to determine the amount required to produce consistent results. For this purpose, RNA ranging in concentration from 20 ng to 100 ng will be tested by hybridization on the Illumina array. Each sample will be analyzed in duplicate to determine the reproducibility of the profiling assay. The minimal amount of RNA required to give rise to a R² value of 0.9 or above in technical repeats will be determined. Biological repeats will also be performed using adjacent FFPE sections using the same criterion. Based upon these experiments a decision on the choice of RASL or DASL for large-scale oligonucleotide synthesis to cover most transcripts in the human genome will be made. Once the strategy is decided, all oligonucleotides will be synthesized and the experiments repeated to document the technology at the genome scale. Based on the titration experiments, the minimal amount of RNA required for gene expression profiling can be determined using existing technology.

Recent development of massive sequencing technologies offers a promising solution to analyze RNA that is insufficient for microarray analysis. Two such sequencing platforms are now commercially available, one from 454 Inc. and the other from Solexa. Both platforms utilize a similar strategy to amplify single DNA molecule tethered either to beads (454 Inc.) or to a solid surface (Solexa) coated with a capture oligonucleotide probe. Both platforms require conversion of dsDNA into ssDNA, each molecule of which is flanked by two unique universal primers. While both platforms can generate a huge number of sequencing reads (˜0.4 million on the 454 platform and ten to hundreds of million on the Solexa platform) in a signal run. It is striking to note that the RASL/DASL products (ligated oligonucleotide pairs) are exactly configured for the massive sequencing strategy on either platform. By coupling DASL with massive sequencing, then, it may be possible to count the number of molecules from individual profiling experiments to obtain quantum information on gene expression.

The 454 platform uses capture of single DNA molecule per bead for subsequent emulsion PCR, which requires input DNA to be carefully titrated before loading onto the beads. In contrast, the Solexa platform uses a primer-coated surface to capture input DNA for local PCR amplification similar to the Polony assay, the technology that can accommodate a wide range of initial DNA concentration and thus has the capacity to produce hundreds of million sequences per run. The disclosure provides a sequencing-based gene expression profiling technology by coupling RASL/DASL assays with sequencing on a 454 Inc. or Solexa platform.

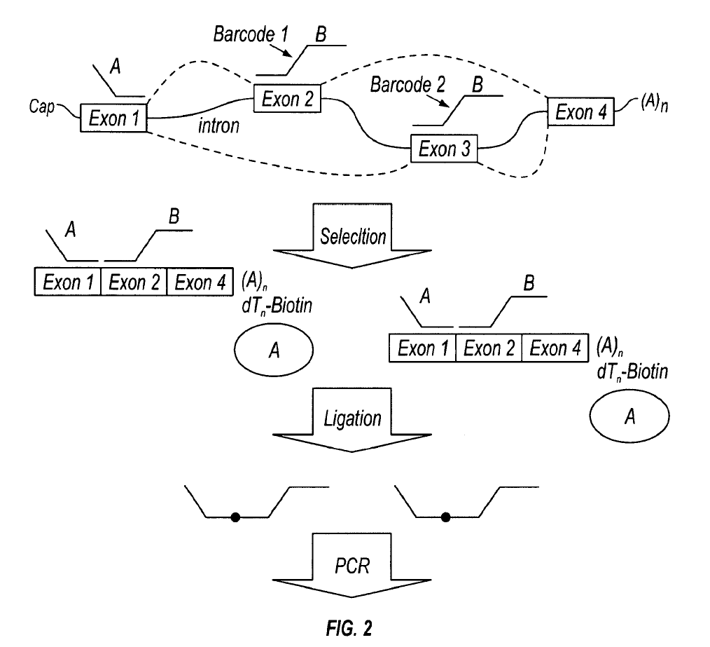

As diagrammed in FIG. 2A, incoming ssDNA becomes trapped individually to the surface of the sequencing slide coated with the capture primer (primer A). Annealed DNA will then template extension of the anchoring primer so that the extended DNA now carries the sequence complementary to anther capture primer (primer B), which is also anchored on the slide surface. Subsequent denaturing allows the extended DNA to snap back and anneal to an adjacent primer B to initiate the next round of extension. The process is repeated, which will produce a colony, the size of which corresponds to the length of the initial DNA. Because the RASL/DASL products are uniform in length, the configuration is ideal to generate colonies that are uniform in size and shape. Each colony will have sufficient number of homogenous DNA for sequencing using either primer A or B.

The RASL/DASL oligonucleotide can be designed using primer A and B in each end so that the products can be directly loaded onto the slide for sequencing. However, to best utilize the sequencing capacity as described below, oligonucleotides will be designed in the original configuration in which the unique sequence is flanked by a different pair of universal primers (C and D). This will allow two rounds of primer extension to be conducted of the initial RASL/DASL products using two fusion primers (A/C and B/D) before loading them onto the sequencing slide. As illustrated in FIG. 2B, one of the fusion primer will be biotinylated. After two rounds of primer extension, the products will be purified by streptavidin selection. The extended products can be released under alkaline condition for loading onto the sequencing slide.

To demonstrate the ability to obtain quantum information on gene expression, an oligonucleotide pool targeting ˜100 genes by multiple oligonucleotide pairs will be used. RASL/DASL will be conducted and then extend the products using universal fusion primers described herein. The oligonucleotide set for each gene will report the average copy number of that gene. Because the hybridization efficiency varies in a large range (2 to 3 orders of magnitude), different oligonucleotides on standard microarrays are known to yield highly variable signal intensities under non-saturating conditions. In contrast to standard hybridization on microarrays, oligonucleotide annealing in RASL/DASL assays is taking place under a saturating condition, and thus, the products may have less variation from different oligonucleotide pairs. In the absence of further signal amplification, quantification of the RASL/DASL products by massive sequencing avoids biases in conventional quantification by hybridization on a microarray. Based on this rationale, the quantum information from different oligonucleotide pairs targeting to different regions in single genes will likely be quite uniform. Under such conditions single oligonucleotide pairs can be used to target individual gene transcripts in the human genome. The multiple oligonucleotide pairs designed for an initial pilot study can be used as internal controls in future full transcriptome analysis to judge the quality of data from individual experiments.

Based on the technology design and the ability to analyze single molecules by sequencing the disclosure provides a sensitivity and accuracy to report gene expression in cells in a quantitative fashion. As described herein RNA preparations are selected that have sufficient concentration for both profiling on microarray and validation by qPCR. Additional aliquots from those samples can be used for the sequencing-based analysis to demonstrate which yields more reliable results in comparison to those individually determined by qPCR. The sequencing-based strategy describe herein will give rise to the most robust data on gene expression.

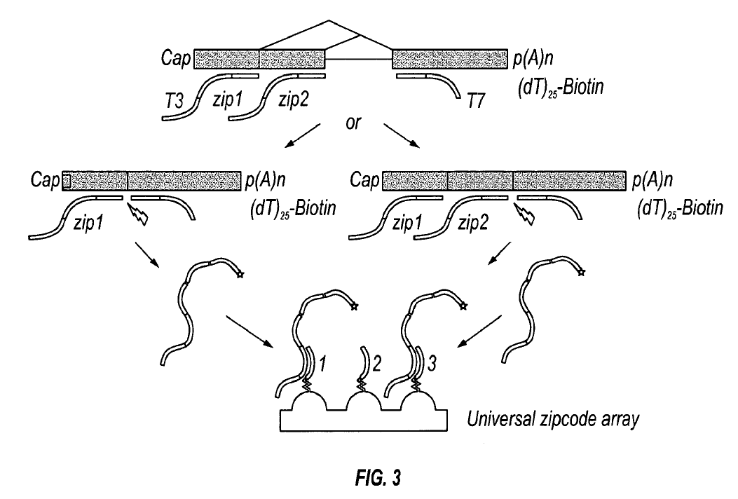

As diagrammed in FIG. 3, 16 versions of fusion primer A/C will be synthesized in which two nucleotides between A and C will be unique in each fusion primer. This will allow the conversion of RASL/DASL products from individual samples using distinct fusion primers in combination with the common B/D primer. Individual pools can then be combined for a single sequencing run to produce data on multiple assays (16 being the maximal number in this design). Because a total of ˜20,000 oligonucleotide pairs (unique pairs per gene plus multiple pairs used to target a limited number of genes for internal controls) are used to target most annotated transcripts in the human genome, an average of 1000 counts per transcript should give sufficient quantitative information on gene expression in each sample, which will require 20 million sequence reads, which is well within the range of the Solexa platform. Using the methods of the disclosure, the cost per sequencing will be reduced to ˜$25 per assay, which is more economical than any microarray platforms on the market, not mentioning the ability to analyze RNA samples that are insufficient for standard microarray analysis. The strategy of the disclosure has the potential to replace microarray-based approaches altogether.

One of the major problems in studying gene expression in cancer is heterogeneous cellular content in most tumor samples. In a given tissue section, tumor cells in different malignant stages may co-exist, which are frequently surrounded by normal stromal cells. As a result, a profile of gene expression from a tissue section will heavily depend on the tumor content in each biological sample or section. One solution to this problem is to isolate pathologically defined tumor cells by laser capture microscopy. While powerful, the isolated material is often insufficient for comprehensive molecular analysis. The disclosure provides a general strategy to conduct quantum analysis of gene expression in a defined cell population without RNA isolation and signal amplification by coupling RASL/DASL with laser capture and massive sequencing.

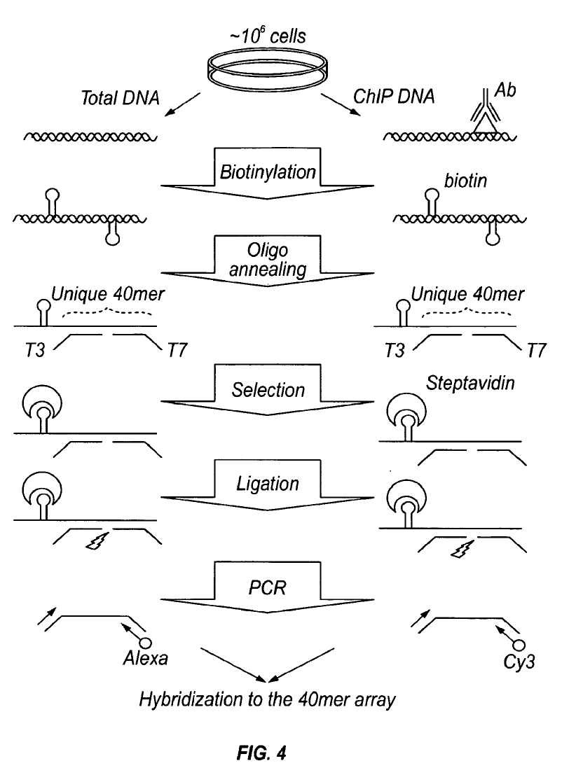

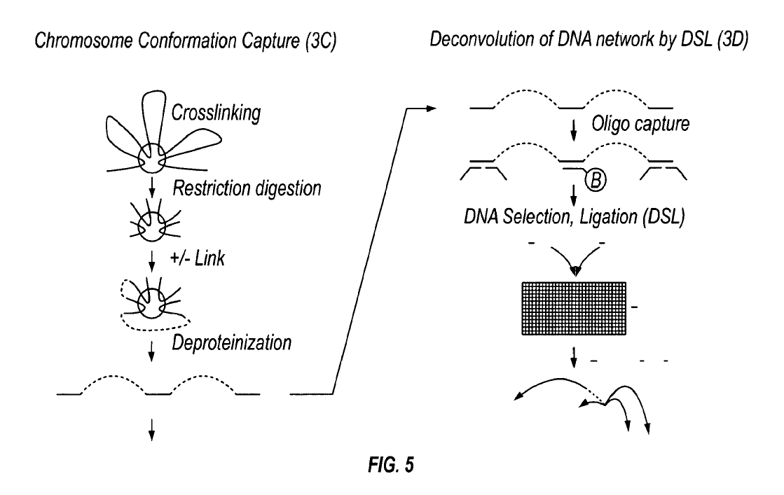

In this aspect, a biological sample (e.g., a tissue sample or section) is prepared as described herein. Oligonucleotides are designed based upon suspected or known gene sequences. As illustrated in FIG. 4, an oligonucleotide pool targeting individual genes in the human genome will be used to perform in situ hybridization on a tissue slide or section. Note that this oligonucleotide pool will be the same as those used in RASL assay, which is one reason to develop the coupling between RASL and the sequencing-based approach to gene expression.

Polynucleotides in a sample are contacted with a primer pair under conditions whereby the primer pair hybridizes to the polynucleotide to form a first hybridization complex, each primer comprising at least two portions, a first portion comprising a target-specific oligonucleotide that is capable of hybridizing to a target polynucleotide, and a second portion comprising a universal primer landing site, the two primers are designed to be specific for an upstream and downstream segment of a polynucleotide, one primer of the pair of primers comprising a first universal primer landing site and the second primer comprising a second universal primer landing site, wherein the universal landing sites are not the same. The first hybridization complex is contacted with a ligase under conditions whereby primer pairs hybridized to the polynucleotide fragment are ligated to form a ligated probe. The ligated probes are amplified with universal primers to generate an amplified-labelled product.

A plurality of probes (also referred to herein as “hybridization probes”) comprise at least two portions: a first portion comprises a target-specific oligonucleotide that is capable of hybridizing to a target polynucleotide, and a second portion comprising a “universal primer landing site”. Two different hybridization probes are designed to be specific for an upstream and downstream segment of a target polynucleotide. An upstream hybridization probe will comprise a first universal primer landing site and the downstream hybridization probe will comprise a second universal primer landing site. The first and second universal landing sites are not the same. Examples of universal primer landing sites include the T7 and T3 universal primer landing sites. In one aspect of the disclosure, the first universal primer landing site is a T7 primer landing site and the second universal primer landing site is a T3 primer landing site.

These hybridization probes are hybridized to the polynucleotides, without prior amplification, to form a first hybridization complex. Probes and primers of the disclosure are designed to have at least a portion be substantially complementary to a target polynucleotide, such that hybridization of the target polynucleotide and the probes or primers of the disclosure occurs. As outlined below, this complementarity need not be perfect; there may be any number of base pair mismatches which will interfere with hybridization between the target polynucleotide and the single stranded hybridization probe of the disclosure. Thus, by “substantially complementary” herein is meant that the probes are sufficiently complementary to the target polynucleotide to hybridize under moderate to high stringency conditions.

After hybridization, free (i.e., non-hybridized) oligonucleotides will be washed away and annealed oligonucleotides will be ligated with a ligase (e.g., a T4 ligase), which is able to catalyze DNA ligation templated by RNA. This process is similar to the RASL scheme except that the ligation reaction is carried out in situ, instead of on beads. After ligation, the slide will be H&E stained for morphological analysis and then mounted to a laser capture microscope to identify a specific cell type(s) to be analyzed. Ligated oligonucleotides associated with the captured tissue will be eluted in water and then processed for sequencing (e.g., sequencing on a 454 or Solexa platform). This experimental strategy, which is referred to as “in situ RASL,” has the potential to obtain quantitative information on gene expression in a defined cell population without RNA isolation and signal amplification.

μCut laser capture microscope are known in the art. Techniques for oligonucleotide-based in situ hybridization on tissue sections are also known in the art. Studies have demonstrated that ˜1000 cells from prostate cancer tissue can be captured by laser capture and that RNA extracted from the captured material is sufficient to conduct a number of PT-PCR analysis of several identified prostate cancer biomarkers. Biotinylated oligonucleotide probes for a number of well-documented breast cancer tumor makers (such ER and Her2) can be used to conduct standard in situ hybridization to work out experimental conditions to show elevated gene expression of those markers in cancer cells in comparison to surrounding stromals. Using this information as a basis, full-scale in situ RASL can be performed to demonstrate the methods of the disclosure. For example, capture of ˜1000 tumor cells and an equivalent amount of stromal cells for signal amplification by PCR using a pair of universal primers followed by hybridization on the illumina arrays can be used. Data from in situ RASL can be compared with those from standard array analysis using isolated RNA as well as those reported in literature.

Using the methods of the disclosure one can quantify gene expression by large-scale sequencing on platforms similar to or including the 454 and the Solexa platform and compare the data with those from microarray analysis. The methods of the disclosure are applicable to addressing a wide range of biological questions in development and disease.

The assays are generally run under stringent conditions, which allows formation of the first hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, and combinations thereof.

These parameters may also be used to control non-specific binding, as is generally outlined in U.S. Pat. No. 5,681,697. Thus it may be desirable to perform certain steps at higher stringency conditions to reduce non-specific binding.

A variety of hybridization conditions may be used in the disclosure, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the polyadenylated mRNA target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of helix destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Complementary oligonucleotide can be immobilized on a glass slide (e.g., Corning Microarray Technology (CMT™) GAPS™) or on a microchip. Conditions of hybridization will typically include, for example, high stringency conditions and/or moderate stringency conditions. (See e.g., pages 2.10.1-2.10.16 (see particularly 2.10.8-11) and pages 6.3.1-6 in Current Protocols in Molecular Biology). Factors such as probe length, base composition, percent mismatch between the hybridizing sequences, temperature and ionic strength influence the stability of hybridization. Thus, high or moderate stringency conditions can be determined empirically, and depend in part upon the characteristics of the polynucleotide (DNA, RNA) and the other nucleic acids to be assessed for hybridization. Generally, stringent conditions are selected to be about 5-10° C. lower than the thermal melting point (Tₘ) for the specific sequence at a defined ionic strength and pH. The Tₘ is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts), at pH 7.0 to 8.3 and the temperature is at least about 30° C. for short probes (e.g., 10 to about 50 nucleotides) and at least about 60° C. for long probes (e.g., greater than about 50 nucleotides). Stringent conditions may also be achieved with the addition of destabilizing agents such as formamide. For selective or specific hybridization, a positive signal (e.g., identification of a nucleic acid) is about 2 times background hybridization. For the purpose of this disclosure, moderately stringent hybridization conditions mean that hybridization is performed at about 42° C. in a hybridization solution containing 25 mM KPO₄ (pH 7.4), 5×SSC, 5×Denhardt's solution, 50 μg/mL denatured, sonicated salmon sperm DNA, 50% formamide, 10% Dextran sulfate, and 1-15 ng/mL probe, while the washes are performed at about 50° C. with a wash solution containing 2×SSC and 0.1% sodium dodecyl sulfate. Highly stringent hybridization conditions mean that hybridization is performed at about 42° C. in a hybridization solution containing 25 mM KPO4 (pH 7.4), 5×SSC, 5×Denhardt's solution, 50 μg/mL denatured, sonicated salmon sperm DNA, 50% formamide, 10% Dextran sulfate, and 1-15 ng/mL probe, while the washes are performed at about 65° C. with a wash solution containing 0.2×SSC and 0.1% sodium dodecyl sulfate.

The size of the primer and probe may vary, as will be appreciated by those in the art with each portion of the probe and the total length of the probe in general varying from 5 to 500 nucleotides in length. Each portion is between 10 and 100, between 15 and 50 and from 10 to 35 being typically used depending on the use and amplification technique. Thus, for example, the universal priming sites of the probes are each about 15-25 nucleotides in length, with 20 being used most frequent. The adapter sequences of the probes are from 5-25 nucleotides in length, with 10-20 being most common. The target specific portion of the probe is typically from 15-50 nucleotides in length, with from 20 to 40 being most common.

Accordingly, the disclosure provides a first hybridization probe set. By “probe set” herein is meant a plurality of hybridization probes that are used in a particular multiplexed assay. In this context, plurality means at least two, but can include more than 10, depending on the assay, sample and purpose of the test.

Accordingly, the disclosure provides hybridization probe sets that comprise universal priming sites. By “universal priming site” herein is meant a sequence of the probe that will bind a PCR primer for amplification. Each probe set comprises an upstream universal priming site (UUP) and a downstream universal priming site (DUP). Again, “upstream” and “downstream” are not meant to convey a particular 5′-3′ orientation, and will depend on the orientation of the system. Typically, only a single UUP sequence and a single DUP sequence is used in a probe set, although as will be appreciated by those in the art, different assays or different multiplexing analysis may utilize a plurality of universal priming sequences. In addition, the universal priming sites are typically located at the 5′ and 3′ termini of the hybridization probe set (or the ligated probe), as only sequences flanked by priming sequences will be amplified.

In addition, universal priming sequences are generally chosen to be as unique as possible given the particular assays and host genomes to ensure specificity of the assay. In general, universal priming sequences range in size from about 5 to about 35 basepairs, with from about 15 to about 20 being typical.

As will be appreciated by those in the art, the orientation of the two priming sites is different. That is, one PCR primer will directly hybridize to the first universal priming site, while the other PCR primer will hybridize to the complement of the second universal priming site. Stated differently, a first universal priming site is in sense orientation, and a second universal priming site is in antisense orientation.

In addition to the universal priming sites, each hybridization probes of the probe set comprise at least a first target-specific sequence. As will be appreciated by those in the art, the target-specific sequence may take on a wide variety of formats, depending on the use of probe. For example through a primer selection program, a specific 40-mer oligonucleotides can be selected to represent a given region (such as promoter) in the human genome. The process will verify its uniqueness by allowing at least 4 evenly distributed mismatches in related sequences in the genome after the BLAST search against the human genome database(s). Selected sequences also avoid small repeats, have a Tm in a defined range (e.g., between about 55 and 65° C.), and contain minimized secondary structure (calculated by ΔG). In parallel, amino-derived oligonucleotides will be synthesized and spotted onto a substrate (e.g., a Motorola 3D codelink slide) to form an oligonucleotide-based array (e.g., a promoter array). The oligomer is essentially split in two (e.g., where the oligomer is a 40-mer it is split in two to provide two 20-mers) to provide target specific sequences that are combined with universal primers and thus become the upstream and downstream hybridization probes.

In this embodiment, the hybridization probes comprise at least a first hybridization probe and a second hybridization probe. The method is based on the fact that two probes can be ligated together, if they are hybridized to a target polynucleotide and if perfect complementarity exists at the junction between the two probes, this does not mean that perfect complementarily must exist across the full length of both probes.

In one embodiment, the two hybridization probes are designed each with a target specific portion. The first hybridization probe is designed to be substantially complementary to a first target domain of a target polynucleotide (e.g., a polynucleotide fragment) and the second hybridization probe is substantially complementary to a second target domain of a target polynucleotide (e.g., a polynucleotide fragment). In general, each target specific sequence of a hybridization probe is at least about 5 nucleotides long, with sequences of about 15 to 30 being typical and 20 being especially common. In one embodiment, the first and second target domains are directly adjacent, e.g., they have no intervening nucleotides. In this embodiment, at least a first hybridization probe is hybridized to the first target domain and a second hybridization probe is hybridized to the second target domain. If perfect complementarity exists at the junction, a ligation structure is formed such that the two probes can be ligated together to form a ligated probe. If this complementarity does not exist, no ligation structure is formed and the probes are not ligated together to an appreciable degree. This may be done using heat cycling, to allow the ligated probe to be denatured off the target polynucleotide such that it may serve as a template for further reactions. The method may also be done using three hybridization probes or hybridization probes that are separated by one or more nucleotides, if dNTPs and a polymerase are added (this is sometimes referred to as “Genetic Bit” analysis).

In one embodiment, the two hybridization probes are not directly adjacent. In this embodiment, they may be separated by one or more bases. The addition of dNTPs and a polymerase are used to “fill in” the gap, followed by the ligation reaction. This allows the formation of the ligated probe.

As will be appreciated by those in the art, nucleic acid analogs find use as primers and probes in the disclosure. In addition, mixtures of naturally occurring nucleic acids and analogs can be made. Alternatively, mixtures of different nucleic acid analogs, and mixtures of naturally occurring nucleic acids and analogs may be made. For example, locked nucleic acids (LNAs) and peptide nucleic acids (PNA) which includes peptide nucleic acid analogs can be used. PNA backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tm for an internal mismatch. With the non-ionic PNA backbone, the drop is closer to 7-9° C. Similarly, due to their non-ionic nature, hybridization of the bases attached to these backbones is relatively insensitive to salt concentration.

A hybridization probe or primer may contain any combination of deoxyribo- and ribo-nucleotides, and any combination of bases, including uracil, adenine, thymine, cytosine, guanine, inosine, xathanine hypoxathanine, isocytosine, isoguanine, and the like. In one embodiment, isocytosine and isoguanine are used in primers and probes as this reduces non-specific hybridization, as is generally described in U.S. Pat. No. 5,681,702. As used herein, the term “nucleoside” includes nucleotides as well as nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

Following ligation, the non-hybridized, hybridization and ligated probes are then removed. In one aspect, this is accomplished by using a streptavidin support that can specifically retain all biotinylated DNA, including hybrid complexes. For example, in one aspect the polynucleotides of a sample are biotinylated prior to being contacted with the hybridization probes. Thus, prior to, during, or after contact with the hybridization probes the biotinylated polynucleotides undergo solid phase selection by contacting the biotinylated polynucleotide with a streptavidin substrate.

In one aspect, once the unhybridized probes are removed, the hybrids are subjected to ligation. The ligated probes can then be used in a sequencing process such as those marketed and used by 454 Inc. and Solexa.

As will be appreciated by those in the art, polynucleotides can be obtained from samples including, but not limited to, bodily fluids (e.g., blood, urine, serum, lymph, saliva, anal and vaginal secretions, perspiration and semen) of virtually any organism, with mammalian samples common to the methods of the disclosure and human samples being typical. The sample may comprise individual cells, including primary cells (including bacteria) and cell lines including, but not limited to, tumor cells of all types (particularly melanoma, myeloid leukemia, carcinomas of the lung, breast, ovaries, colon, kidney, prostate, pancreas and testes); cardiomyocytes; endothelial cells; epithelial cells; lymphocytes (T-cell and B cell); mast cells; eosinophils; vascular intimal cells; hepatocytes; leukocytes including mononuclear leukocytes; stem cells such as haemopoetic, neural, skin, lung, kidney, liver and myocyte stem cells; osteoclasts; chondrocytes and other connective tissue cells; keratinocytes; melanocytes; liver cells; kidney cells; and adipocytes. Suitable cells also include known research cells, including, but not limited to, Jurkat T cells, NIH3T3 cells, CHO, Cos, 923, HeLa, WI-38, Weri-1, MG-63, and the like (see the ATCC cell line catalog, hereby expressly incorporated by reference).

A target polynucleotide includes a polymeric form of nucleotides at least 20 bases in length. An isolated polynucleotide is a polynucleotide that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an automatically replicating plasmid or virus; or into the genomic DNA of a prokaryote or eukaryote, which exists as a separate molecule (e.g., a cDNA) independent of other sequences, as well as genomic fragments that may be present in solution or on microarray chips. The nucleotides of the disclosure can be ribonucleotides, deoxyribonucleotides, or modified forms of either nucleotide. The term includes single and double stranded forms of DNA.

The term polynucleotide(s) generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions.

In addition, polynucleotide also includes triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

In some aspects a polynucleotide or oligonucleotide (e.g., a probe, a primer or primer pair) includes DNAs or RNAs as described above that contain one or more modified bases. Thus, DNAs or RNAs with backbones modified for stability or for other reasons are nucleic acid molecules. Moreover, DNAs or RNAs comprising unusual bases, such as inosine, or modified bases, such as tritylated bases, to name just two examples, are polynucleotides or oligonucleotides as the term is used herein.

It will be appreciated that a great variety of modifications have been made to DNA and RNA that serve many useful purposes known to those of skill in the art. Polynucleotides and oligonucleotides include such chemically, enzymatically or metabolically modified forms of polynucleotides, as well as the chemical forms of DNA and RNA characteristic of viruses and cells, including simple and complex cells, inter alia.

Components of the reaction may be added simultaneously, or sequentially, in any order, with typical embodiments. In addition, the reaction may include a variety of other reagents which may be included in the assays. Such other reagents include salts, buffers, neutral proteins, e.g. albumin, detergents, and the like, which may be used to facilitate optimal hybridization and detection, and/or reduce non-specific or background interactions. Also reagents that otherwise improve the efficiency of the assay, such as protease inhibitors, nuclease inhibitors, anti-microbial agents, and the like, may be used, depending on the sample preparation methods and purity of the polynucleotides.

In general, PCR may be briefly described as follows. A double stranded hybridization complex is denatured, generally by raising the temperature, and then cooled in the presence of an excess of a PCR primer, which then hybridizes to a universal priming site (e.g., a T7 or T3 priming site). A DNA polymerase then acts to extend the primer with dNTPs, resulting in the synthesis of a new strand forming a hybridization complex. The sample is then heated again, to disassociate the hybridization complex, and the process is repeated. By using a second PCR primer for the complementary target strand that hybridizes to the second universal priming site, rapid and exponential amplification occurs. Thus, PCR steps are denaturation, annealing and extension. The particulars of PCR are well known, and include the use of a thermostable polymerase such as Taq I polymerase and thermal cycling. Suitable DNA polymerases include, but are not limited to, the Klenow fragment of DNA polymerase I, SEQUENASE 1.0 and SEQUENASE 2.0 (U.S. Biochemical), T5 DNA polymerase and Phi29 DNA polymerase. The polymerase can be any polymerase, but typically will lack 3′ exonuclease activity. Examples of suitable polymerase include but are not limited to exonuclease minus DNA Polymerase I large (Klenow) Fragment, Phi29 DNA polymerase, Taq DNA Polymerase and the like. In addition, in some embodiments, a polymerase that will replicate single-stranded DNA (i.e. without a primer forming a double stranded section) can be used.

The reaction is initiated by introducing the ligated probe to a solution comprising a universal primer, a polymerase and nucleotides. A nucleotide is a deoxynucleoside-triphosphate (also called deoxynucleotides or dNTPs, e.g. dATP, dTTP, dCTP and dGTP). In some embodiments, as outlined below, one or more of the nucleotides may comprise a detectable label, which may be either a primary or a secondary label. In addition, the nucleotides may be nucleotide analogs, depending on the configuration of the system. Similarly, the primers may comprise a primary or secondary label.

Accordingly, the PCR reaction requires at least one and typically two PCR primers, a polymerase, and a set of dNTPs. As outlined herein, the primers may comprise the label, or one or more of the dNTPs may comprise a label.

These embodiments also have the advantage that unligated probes need not necessarily be removed, as in the absence of the target, no significant amplification will occur. These benefits may be maximized by the design of the probes; for example, in the first embodiment, when there is a single hybridization probe, placing the universal priming site close to the 5′ end of the probe since this will only serve to generate short, truncated pieces in the absence of the ligation reaction.

By “label” or “detectable label” is meant a moiety that allows detection. This may be a primary label or a secondary label. Accordingly, detection labels may be primary labels (i.e. directly detectable) or secondary labels (indirectly detectable).

In one embodiment, the detection label is a primary label. A primary label is one that can be directly detected, such as a fluorophore. In general, labels fall into three classes: a) isotopic labels, which may be radioactive or heavy isotopes; b) magnetic, electrical, thermal labels; and c) colored or luminescent dyes. Labels can also include enzymes (e.g., horseradish peroxidase, and the like) and magnetic particles. Common labels include chromophores or phosphors but are typically fluorescent dyes. Suitable dyes for use in the disclosure include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”), pyrene, Malacite green, stilbene, Lucifer Yellow, Cascade Blue™, Texas Red, Cy dyes (Cy3, Cy5, and the like), alexa dyes, phycoerythin, bodipy, and others described in the 6th Edition of the Molecular Probes Handbook by Richard P. Haugland, hereby expressly incorporated by reference.

A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, and the like. Secondary labels include, but are not limited to, one of a binding partner pair such as biotin/streptavidin; chemically modifiable moieties; nuclease inhibitors; enzymes such as horseradish peroxidase; alkaline phosphatases; luciferases, and the like.

The secondary label is typically a binding partner pair. For example, the label may be a hapten or antigen, which will bind its binding partner. For example, suitable binding partner pairs include, but are not limited to: antigens (such as proteins (including peptides)) and antibodies (including fragments thereof (FAbs, and the like)); proteins and small molecules, including biotin/streptavidin; enzymes and substrates or inhibitors; other protein-protein interacting pairs; receptor-ligands; and carbohydrates and their binding partners. Nucleic acid-nucleic acid binding protein pairs are also useful. In general, the smaller of the pair is attached to a nucleotide for incorporation into the primer. Typical binding partner pairs include, but are not limited to, biotin (or imino-biotin) and streptavidin, digeoxinin and Abs, and Prolinx™ reagents. For example, the binding partner pair can comprise biotin or imino-biotin and a fluorescently labeled streptavidin. Imino-biotin disassociates from streptavidin in pH 4.0 buffer while biotin requires harsh denaturants (e.g., 6 M guanidinium HCl, pH 1.5 or 90% formamide at 95° C.).

Labelling can occur in a variety of ways, as will be appreciated by those in the art. In general, labelling can occur in one of two ways: labels are incorporated into primers such that the amplification reaction results in amplicons that comprise the labels or labels are attached to dNTPs and incorporated by the polymerase into the amplicons.

The amplified DNA can be fluorescently labeled by including fluorescently-tagged nucleotides in the LM-PCR reaction or by fluorescently labelling the universal primers.

Generally, the array will comprise from two to as many as a billion or more different sequences, depending on the size of the substrate as well as the end use of the array. Thus very high density, high density, moderate density, low density and very low density arrays may be used. For example, very high density arrays comprise from about 10,000,000 to about 2,000,000,000 nucleic acid molecules, about 100,000,000 to about 1,000,000,000 being typical (all numbers being in cm2). High density arrays comprise a range of about 100,000 to about 10,000,000 nucleic acid molecules, with about 1,000,000 to about 5,000,000 being typical. Moderate density arrays range from about 10,000 to about 100,000 being typical, and from about 20,000 to about 50,000 being most common. Low density arrays generally comprise less than 10,000 nucleic acid molecules, with from about 1,000 to about 5,000 being typical. Very low density arrays comprise less than 1,000 nucleic acid molecules, with from about 10 to about 1000 being typical, and from about 100 to about 500 being most common.

By “substrate” or “solid support” is meant any material that can be modified to contain discrete individual sites appropriate for the attachment or association of oligonucleotides, polynucleotides, or other organic polymers and is amenable to at least one detection method. Possible substrates include, but are not limited to, glass and modified or functionalized glass, plastics (including acrylics, polystyrene and copolymers of styrene and other materials, polypropylene, polyethylene, polybutylene, polyurethanes, Teflon, and the like), polysaccharides, nylon or nitrocellulose, resins, silica or silica-based materials including silicon and modified silicon, carbon, metals, inorganic glasses, optical fiber bundles, and a variety of other polymers. In general, the substrates allow optical detection and do not themselves appreciably interfere with optical detection (e.g., do not fluoresce themselves).

Generally the substrate is flat (planar), although as will be appreciated by those in the art, other configurations of substrates may be used as well. For example, three dimensional configurations can be used, for example by embedding beads in a porous block of plastic that allows sample access to the beads and using a confocal microscope for detection. Similarly, the beads may be placed on the inside surface of a tube for flow-through sample analysis to minimize sample volume.

Generally, the array compositions can be configured in several ways. For example, a first substrate comprising a plurality of assay locations (sometimes also referred to herein as “assay wells”), such as a microtiter plate, is configured such that each assay location contains an individual array. That is, the assay location and the array location are the same. For example, the plastic material of the microtiter plate can be formed to contain a plurality of “wells” in the bottom of each of the assay wells.

In another aspect, the number of individual arrays is set by the size of the microtiter plate used. Thus, 96 well, 384 well and 1536 well microtiter plates utilize composite arrays comprising 96, 384 and 1536 individual arrays, although as will be appreciated by those in the art, not each microtiter well need contain an individual array. It should be noted that the composite arrays can comprise individual arrays that are identical, similar or different. That is, in some embodiments, it may be desirable to do the same 2,000 assays on 96 different samples. Alternatively, doing 192,000 experiments on the same sample (i.e. the same sample in each of the 96 wells) may be desirable. Alternatively, each row or column of the composite array could be the same for redundancy/quality control. As will be appreciated by those in the art, there are a variety of ways to configure the system. In addition, the random nature of the arrays may mean that the same population of beads may be added to two different surfaces, resulting in substantially similar but perhaps not identical arrays.

A signature ˜40 nt sequence is first computationally identified in a genomic segment 0.5 to 1 kb in length. To construct a tiling array, each probe is used to represent a ˜0.5 kb non-repetitive genomic block in a path to be tiled. Amine-modified 40mers can be spotted onto solid support to form an array. In essence, this probe design strategy selects unique sequences in a genome to construct microarrays, thereby minimizing cross hybridization by related sequences, especially repetitive DNA.

Corresponding to each 40mer, a pair of assay oligonucleotides are synthesized, each consisting of the two 20mer halves in the 40mer and flanked by a universal primer-landing site. Multiple oligonucleotide pairs are mixed to form a pool.

### Examples

### 1. Random Priming-Based Technologies

For high throughput sequencing, it is required to generate DNA library in which specific DNA to be sequenced has to be flanked with two specific sequencing primers at the ends. The existing methods for this purpose require linker ligation or ligation to a vector containing the primer sequences. The methods thus involve complicated library construction, and multiple technical challenging steps are not only tedious and time-consuming, but also error prone.

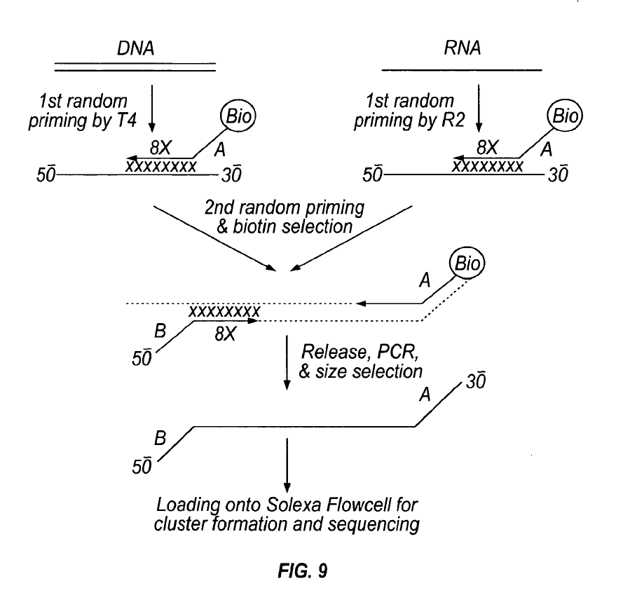

The methods described herein include random priming-based technology as diagrammed in FIG. 9, which significantly simplifies the library construction process for high throughput sequencing. Briefly, a universal primer that includes a random 4 to 20 mer and a sequencing primer (A) at the 5′ end is provided. This primer is optionally includes a capture moiety, such as biotin, at the 5′ end for selection. The primer may be extended by primer extension on DNA (by a DNA polymerase) or RNA (by a reverse transcriptase). At the end of the reaction, ddNTPs are added to block the 3′ ends of extended DNA. Free substrates may be removed with a sizing column. The first random priming products are next used to template the second random priming reaction using another universal primer including a random 4 to 20 mer and a second sequencing primer (B). After the second random priming reaction, the products are selected using a binding partner to the capture moiety, such as streptavidin beads, which provides for the removal of free nucleotide substrates and other small by-products. The products are released from the binding partner by heating or under an alkaline condition. The released single-stranded nucleic acid can be amplified by PCR followed by size selection on agarose gel to isolate amplicoms between 100 to 300 nt in length. The isolated amplicoms are quantified, adjusted to proper concentration, and subjected to high throughput sequencing.

The random priming-based sequencing technology described herein is efficient the selection step removes primer dimers and because A primer-containing products are retained by the binding partner. Accordingly, B primer only products are removed during the selection. Minor contaminations are further removed during size selection on the agarose gel. The PCR amplification step not only increases the sensitivity of the assay but also provides quantification of the products for loading the right amount of DNA to a high throughput sequencer.

The use of random primers provides the advantage of using maximal PCR amplification without concern for biases during the step. It is unlikely that the same random sequence lands on the same DNA or RNA during the random priming reactions. In contrast, PCR products from single DNA or RNA will have exact the same sequences. As a result, bioinformatics can be used to filter out PCR amplified products, which will convert the molecular profile to the original representation. This feature is useful for many applications described throughout this application, particularly during profiling of gene expression in cells.

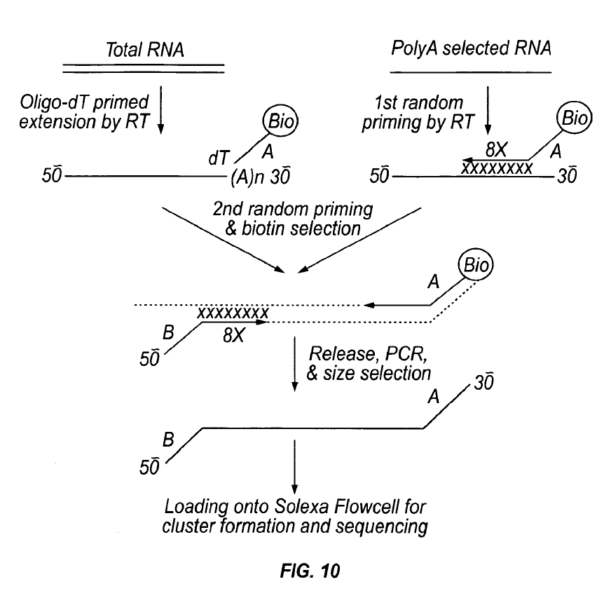

Application of random priming-based sequencing technology n profiling gene expression: The technology can be employed to profile gene expression in various ways. Two exemplary approaches are described in FIG. 10. First, the first universal primer diagrammed in FIG. 9 can be replaced by a primer containing oligo-dT followed by the 5′ sequencing primer. This will allow priming from the 3′ end of polyA mRNA using total RNA. The second priming reaction will be random primed. This strategy is simplified from SAGE-based methods. Because of the PCR amplification step and the ability to convert the sequence information to the original representation, the technology can be used to profile gene expression in a small number of cells, perhaps single cells.

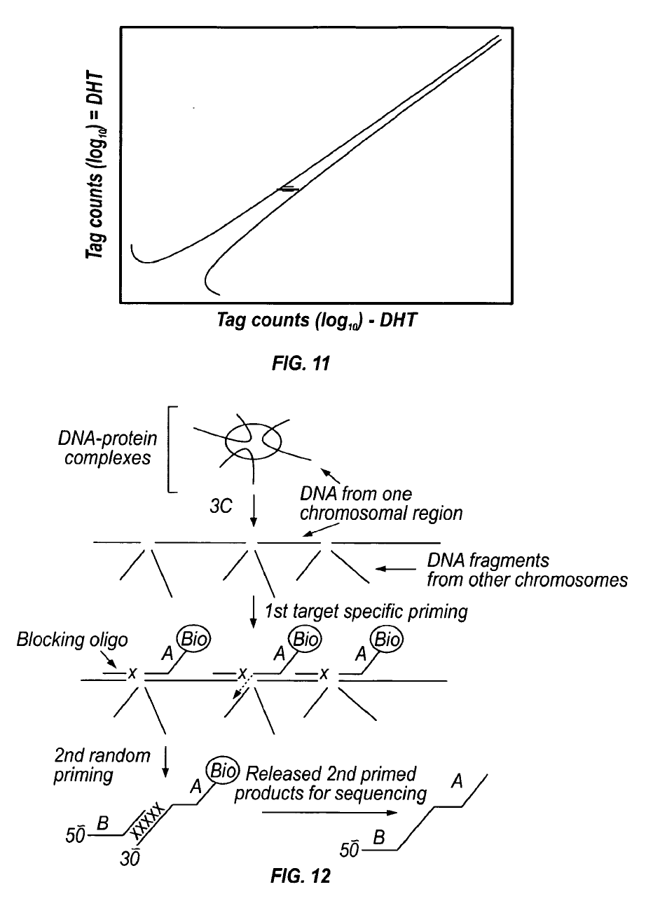

The second exemplary strategy utilizes double random primers. In this case, purified polyA mRNA may be used for profiling gene expression to avoid the interference of rRNA and tRNA in total RNA. The advantage of this method is that it will allow detection of sequence tags distributed along transcripts, making it possible to detect alternative 5′ exons, alternative splicing in gene body, and alternative 3′ ends. Thus, the technology is suitable for complete transcriptome mapping in the cell. FIG. 11 depicts the results of a series of experiments using the double random priming method described above.

Application of random priming-based sequencing technology in mapping DNA-protein interactions in vivo: The double random priming approach can be coupled with chromatin immunoprecipitation (ChIP) to map genome-wide DNA-protein interactions. The technology may be used to map histone medications and DNA binding transcription factors in various cell types.

Application of random priming-based sequencing technology in detecting long-distance DNA-DNA interactions: The technology can be coupled with the conventional 3C (chromosome conformation capture) assay to detect intra- and inter-chromosomal interactions. In this application, a 3C assay is performed on a chosen cell type, which will detect DNA that are tethered by protein complexes in vivo, resulting DNA shuttling after ligation. The DNA is next extracted. Two exemplary strategies were devised to map DNA-DNA interactions as diagrammed in FIG. 12. One is target-specific and the other is global.

As depicted in FIG. 12, the target specific method employs a set of sequence specific primers, each linked to the universal sequencing primer (A) to target a specific DNA region. The annealed primers are extended to shuttled DNA (shaded lines for DNA from other regions in the same chromosomes or different chromosomes) in the 3C reaction. To minimize the interference of religated DNA in the original configuration, the corresponding primers (blocking primers) were used to block the extension of the religated DNA. The extended products are next used to template the random priming reaction using the universal primer consisting of random 4 to 20 mer associated with the other sequencing primer (B). This will allow mapping of long-distance DNA-DNA interactions in the cell to map gene networks critical for the organization of the nucleus and regulated gene expression.

A true global mapping will be the replace the first target specific primer set with the universal primer consisting of random 4 to 20 mer associated with the sequencing primer (A). In this approach, a much larger number of sequence tags is likely required to obtain quantitative results to map DNA networks in the cell.

Application of random priming-based sequencing technology for mapping genome variations, including deletion, insertion, amplification, retrotransposition, and chromosome translocation: The double random priming technology described in FIG. 9 can be applied to study genomic changes by mapping both ends of each amplicoms. This will allow detection of DNA deletion, insertion, retrotransposition, and chromosome translocation events in comparison to sequenced genomes. The quantitative differences may also allow detection of gene amplifications and chromosome losses in cancer cells.

Application of random priming-based sequencing technology for studying the biological function of repetitive sequences in the genome: By sequencing both ends of each amplicoms, it is also possible to use the technology to map repetitive elements in the genome by taking advantage of their boarding with specific genomic sequences. This type of studies may provide critical insights into the organization of the nucleus in eukaryotic cells.

### 2. Oligo Ligation Selection-Based Technologies

The sequencing based method described above is suitable for unbiased detection of gene expression or other genetic variations at the DNA or RNA level. However, low abundant messages may be overpowered by high abundant messages. As a result, sequence tags for low abundant messages may not be sufficient for quantification purposes. This limitation is of a particular concern for profiling alternative splicing. To address this problem and develop complementary approaches, provided herein are modified versions of RNA or DNA Annealing, Selection, and Ligation (RASL/DASL) approaches for quantification by sequencing instead of by microarrays.

As depicted in FIG. 2, the RASL/DASL approaches described herein have been modified to incorporate features for high throughput sequencing. For a given DNA or RNA sequence, a pair of oligos are used to target the sequence. Each oligo includes a terminal universal primer. After annealing and selection via randomly attached capture moiety to DNA or RNA (through the use of a heat-activated biotinylation system), nucleic acids bound to a binding partner are treated with ligase to link paired primers, which converts half-amplicoms to full amplicoms. In contrast, oligos that are non-specifically retained to a binding partner, tube well, DNA or RNA remain half amplicoms. In this way, the specificity in hybridization is ensured by requiring specific annealing without gap between two oligos and ligation of the oligos that are paired without any gap. PCR may be used to amplify the ligated products. The products may be quantified by techniques known to the skilled artisan. In some aspects a specific sequence (zipcode) may be linked to one of the oligos in the pair so that the PCR amplified products can be quantified on the universal zipcode array. However, existing zipcode arrays are generally limited to ˜1500 unique zipcodes which permits detection of only 1500 genes or mRNA isoforms.

By couple the modified RASL/DASL approach with random priming-based sequencing technology the zipcode component is no longer necessary. This not only lowers the cost of manufacturing an oligonucleotide, but also allows for the use of a much larger pool of oligos to target the majority of annotated genes or mRNA isoforms. For example, the PCR amplified products resulting from a RASL/DASL assay can be directly subjected to high throughput sequencing to obtain the digital information on gene expression and/or alternative splicing, which eliminates potential biases introduced during hybridization on microarrays.

Application of the technology in studying low copy gene expression: By coupling RASL with high throughput sequencing, low abundant gene products can be selectively targeted, therefore reducing or eliminating potential interference by high abundant gene products. Because oligo annealing takes place with excess oligos, such saturating condition provides pseudo-first order kinetics during oligo hybridization on RNA targets. As a result, the number of paired and ligated oligos will generally be directly proportional to the abundance of their targets. Because direct sequencing practically eliminates potential biases or cross-hybridization on microarrays, the resulting information should be quantitative.

Application of the technology in profiling alternative RNA processing: Similar to the application described above, individual oligos can be designed to target specific annotated mRNA isoforms. This will allow direct and quantitative profiling of alternative RNA splicing.

Application of the technology in analyzing specific genome variations: Oligo pairs may be designed in a tiling fashion against a sequenced genome. The use of such oligos will allow for the detection of genetic variations, including SNPs, genome deletion, loss of heterozygosity (LOH), loss of chromosome arm, etc. by coupling DASL with high throughput sequencing. For example, if one places one pair of oligos every 100 kb along the human genome, 30,000 pairs of oligos will be sufficient to cover the entire human genome. This will allow mapping of potential disease gene at the resolution of ˜0.1 cM. The same oligo pool can also be used to detect large chromosomal deletions and LOH at the resolution.

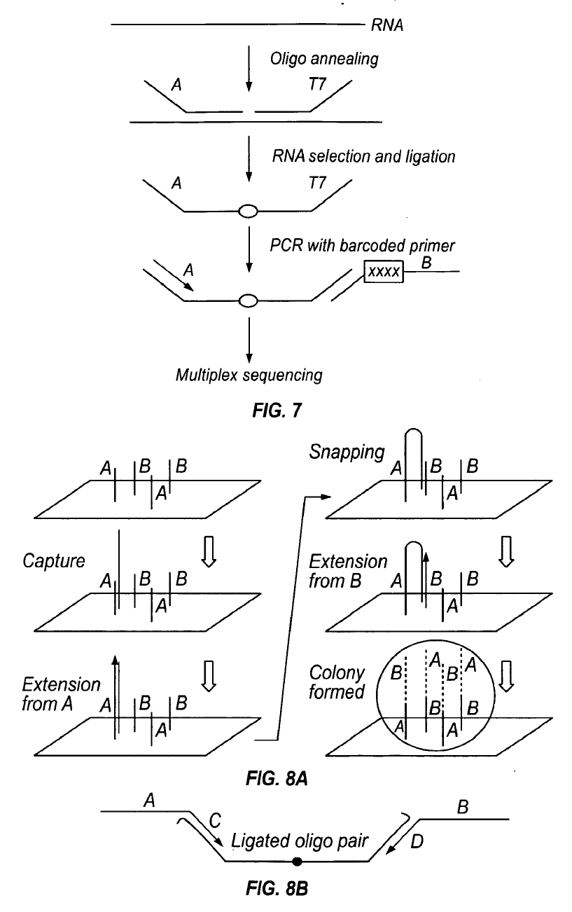

Multiplex strategy for parallel analysis of gene expression signature: Gene expression by microarray or sequencing-based approaches has been widely used in basic and clinical research. However, the throughput is not sufficient for applications where a large number of parallel reactions are required. In fact, in those applications, whole genome information may not be essential. For example, methods provided herein can be used to monitor 100 to 1000 genes in a 96-well format screening by designing oligonucleotide pairs for each target gene in which the 5′ oligo is flanked with a universal sequencing primer (A) and the 3′ oligo is flanked with another universal sequence (e.g. T7 primer), as diagrammed in FIG. 7. After the ligation step as in DASL/RASL, the ligated product can be amplified with a set of primers, each of which contains the common primer (A) and a hybrid primer consisting of T7, a unique 2 to 8 mer as a molecular barcode, and the sequencing primer B. Each primer pair is applied to a well and a robot can handle 96-well amplification in an automated fashion. After PCR amplification, the products from each plate can be pooled for high throughput sequencing from both ends. One end will give the information on specifically targeted genes and the other will identify the unique molecular sequence as the identifier for the sample in the plate. In this way, 96 samples can be profiled for the expression of a panel of genes simultaneously. This approach can be similarly performed on other high throughput formats available to the skilled artisan.

This parallel sequencing technology is suitable for use in drug screening. In most applications, screening in a 384 format is performed. If each plate can be screened in a single reaction, it will be practical to go through a large of candidate drugs or even the entire drug library.

### 3. In Situ Hybridization Coupled Detection of Gene Expression in Defined Cell Types

In another embodiment, the methods described herein can be used to obtain digital information on gene expression, genome wide and in single cells, without RNA isolation, signal amplification, and hybridization on microarrays. The application of these methods to in situ systems will allow determination of changes in gene expression in different cell types, during different developmental stages of special cell lineages, accompanying cancer progression, etc.

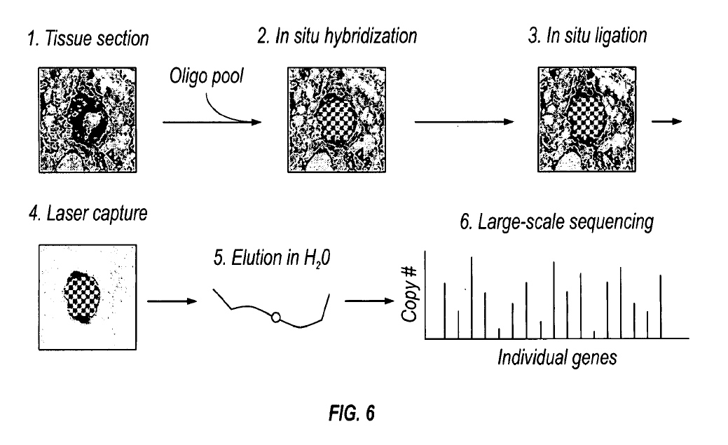

As illustrated in FIG. 6, the first step is to prepare a tissue section or a cell culture coverslip. This step is a routine in most cell and molecular biology labs. A specific region in each gene transcript is selected for targeting by a pair of oligo as in RASL. The oligo pool can be used to perform in situ hybridization on the tissue section or cell coverslip. In situ hybridization is routinely carried out using one or a few oligos in many labs. Free oligos may be washed away after in situ hybridization, and annealed oligos in pairs may be ligated using T4 ligase, converting oligo pairs amplicons that can be amplified by PCR. After the in situ RASL reaction, the tissue may be stained and specific cells isolated from the coverslip by laser capture microscopy. Ligated oligos can be released from captured material and then subjected to PCR amplification and high throughput sequencing.

Applications of the technology: The in situ methods described above permit gene expression profiling in defined cell types without RNA isolation. This will greatly facilitate studies on gene expression in development, identification of biomarkers associated defined lesions (such as cancer), and analysis of molecular programs induced by cell-cell interactions in tissues (such as stroma-cancer interactions). It may be also possible to identify key genes in different locations within a single cell by using this method.

4. Prostate cancer: Prostate cancer is the most common male cancer in the US. Compared to other cancer types, prostate cancer is unique in its late onset, perhaps due to accumulated alterations in the genome, which progressively convert normal prostate epithelial cells to hyper-proliferative neoplasm to androgen-independent tumors. This proposal is propelled by the discovery of gene networks in breast and prostate cancer cells where multiple signaling-dependent genes, such as those regulated by nuclear hormones, are engaging in specific intra- and inter-chromosomal interactions in the nucleus. In light of recent realization of prevalent chromosome translocation events in solid tumors, including prostate cancer, specific gene networking may be a prerequisite for chromosomal translocation in response to genome instability cures in cancer. Accordingly, defects in genetic and epigenetic control of regulated gene expression may directly contribute to the progression of initial androgen sensitive neoplasm to androgen-independent prostate tumors.

Digital analysis of genome rearrangement in prostate cancer: Many studies on sarcomas and carcinomas have raised the possibility that chromosome translocation may be much more common in solid tumors than previously appreciated, suggesting that many more chromosome translocation events remain to be detected and characterized in solid tumors. High throughput sequencing technology as described in the present application can be used to perform global mapping of chromosome translocation events in the prostate cancer model to understand cancer etiology.

Elucidation of gene networking in prostate cancer cells: Based on the recent discovery of gene networks in breast and prostate cancer cells, it is hypothesized that specific intra- and inter-chromosomal interactions are precursors to aberrant chromosomal translocation in tumors. The present methods can be used to develop and perform global gene network analysis through coupling the conventional chromosome conformation capture (3C) assay with high throughput sequencing to elucidate functional intergenic interactions, which may serve as precursor for permanent chromosomal rearrangement in cancer.

Determination of the molecular basis for the development of androgen refractory prostate tumors: Methods provided herein may be used to determine the genetic and epigenetic factors associated with androgen-regulated gene expression. A cellular genetics approach will be used to determine the role of specific histone methyltransferases and demethylases in conferring androgen dependency.

Recurrent chromosome translocation in prostate cancer: Prostate cancer is a late onset disease and second leading cause of male mortality in the US. The development of prostate cancer goes through several transitions from initial neoplasm to more aggressive tumor phenotype, which, like most other tumor types, ultimately acquires the ability to metastasize to distant organs. Similar to breast cancer, prostate cancer is initially sensitive to hormone ablation therapy because neoplastic tumors depend on androgen to proliferate. Because normal prostate epithelial cells also depend on androgen to grow, it has remained a mystery how androgen sensitive tumor cells may arise from normal prostate epithelium.

Gene expression profiling has been widely applied to prostate cancer. Although microarray studies can detect a panel of elevated or suppressed genes, it has been unclear in most cases which gene(s) contributes to a specific tumor phenotype and which drives tumor progression. Recently, a unique bioinformatics approach was developed to detect genes whose expression is dramatically altered only in subsets of prostate tumors, instead of those uniformly induced or repressed in prostate cancer compared to normal prostate issues. The rationale of this powerful “Cancer Outlier Profile Analysis” is that prostate cancer may arise from a spectrum of distinct genetic lesions, which may individually contribute to the onset of disease in a small subset of prostate cancer patients. Strikingly, molecular analysis of several identified outliers revealed a common mechanism for their overexpression in prostate cancer: A specific androgen-regulated gene (TMPRSS2) was found to fuse with multiple members of the ETS gene family (ETV1, ETV4, and ERG) in both prostate cancer cell lines and primary tumors. Because the ETS family genes are known to play critical role in cell growth control, the formation of fusion genes then rendered those cell growth regulatory genes under the androgen control, which provides a plausible mechanism for cells carrying such fusion genes to become hyper-proliferative in the presence of androgen.

These recent advances also emphasize the prevalence of chromosome translocation in solid tumors, which is once thought to be a unique feature of hematological malignancies. Chromosome translocation may result from specific intergenic interactions in regulated gene expression in normal cells. In addition, there appears to be a role for splicing factors in transcriptional elongation, which provides the mechanism for the recent observation that depletion of critical splicing factors trigger widespread double-stranded DNA breaks. The methods provided herein allow for the further elucidation of inter-chromosomal interactions coupled with genomic instability induced by defects in transcription elongation may potentiate specific gene rearrangement events. Accordingly, the present methods provide for the opportunity to identify cancers that may arise from abnormal gene expression rather than errors in DNA replication or DNA damages induced by environmental mutagens.

The activation of oncogenic signal transduction pathways contributes to the hyper-proliferation state of tumor cells, but primary genetic lesions that trigger the activation of these pathways often remain elusive in many different types of tumors. With regard to prostate cancer, the activation of various oncogenic pathways may induce AR expression and/or stimulate the expression of AR target genes. However, it remains to be understood how the AR bypasses the requirement for androgen in cases where there are no mutations in the AR gene itself.

Normal specific inter-chromosomal interactions may be an underlying mechanism for chromosome translocation in cancer. A novel role of splicing factors in transcriptional elongation and double-stranded DNA breaks in response of splicing factor depletion in vivo was observed, suggesting that transcriptional elongation associated defects may be a critical cue for genome instability in cancer. Accordingly, provided herein are methods for profiling alternative splicing in prostate cancer cells and tissues. Also provided are methods for identifying regulated gene expression at both the transcriptional and post-transcriptional levels. As evident in this application, methods of determining the etiology of various cancers rely on high throughput sequencing-based digital technologies provided herein.

The initial goal was to develop a general approach to profiling alternative splicing in development and disease. Alternative splicing represents a form of genetic information shuttling to allow differential incorporation of exonic sequences into final mRNA transcripts. The challenge is to detect highly related mRNA isoforms generated from the same precursor mRNA, which may differ by as small as a few nucleotides or as big as an entire exon of several hundred nucleotides in length. A common approach to detecting mRNA isoforms employs a set of exon junction oligos, which suffer cross-hybridization because mRNA isoforms always share one half junction sequences. To address this problem, an oligo ligation-based approach was developed using two oligos to target a specific exon junction. As diagrammed in FIG. 3, the oligos are all linked to a universal primer. If a specific exon junction exists as a result of alternative splicing, the two oligos will anneal onto the junction next one another, permitting ligation by the T4 ligase, and the ligated oligos can be amplified with the universal PCR primers. This technology is referred to as RNA-mediated Annealing, Ligation, Selection or RASL. To detect individual mRNA isoforms, an index sequence (called zipcode) is linked to each isoform-specific oligo, and the final PCR products can be quantified on a zipcode array (home made or commercially available from Illumina). To support the application of this platform, an alternative splicing database was developed to organize the targets, aid in oligo design, and facilitate data analysis. These methods have been applied to profiling mRNA splicing in prostate cell lines and primary tumors, revealing a panel of cancer-specific mRNA isoform signatures (Li et al., 2006, Cancer Res 66, 4079-4088). In these studies, a concept of characterizing cancer by quantifying transcript changes and mRNA isoform ratios was provided, a procedure referred to as “two-dimensional profiling” (Li et al., 2006, Cancer Res 66, 4079-4088).

One limitation of the zipcode-based approach is the difficulty to enlarge the size of targets that can be interrogated in a single reaction. To overcome this problem, the methods disclosed herein extend the ligation-based approach by making the zipcode optional in the oligo design and printing a separate oligo array for detection. This approach is suitable for genome-wide analysis of DNA-protein interactions by coupling DASL with chromatin immunoprecipitation (ChIP) as illustrated in FIG. 4. In general, signature sequences are computationally selected in each annotated gene promoter in the human genome to construct an oligo-based (40mer) promoter array. Corresponding to each signature sequence, a pair of oligos are designed, each targeting half of the signature sequence. After ligation, the products can be directly hybridized to the oligo array. At least 20,000 pairs of oligos could be pooled for a signal assay without interference. This approach has been used to identify hormone-regulated gene expression, which revealed an unprecedented number of gene promoters that are directly targeted by the estrogen receptor alpha (ERα).

The present methods are based, in part, on array-based and sequencing-based methods for quantitative analysis of gene expression, DNA-protein interactions, and numerous other applications in functional genomics. In some embodiments the methods described herein include converting nucleic acid samples to small amplicoms (100 to 200 nt in length), each of which carries unique universal primers at the end. Accordingly, DNA from ChIP is normally ligated to specific linkers containing the universal primers followed by PCR amplification and size selection in an agarose gel. For gene expression profiling, RNA is processed according to the SAGE protocol followed by size selection. The amplicom mix is then loaded onto, e.g., a Solexa flowcell and each molecule is amplified in situ on the surface of the flowcell to form a large number of clusters for parallel sequencing.

This technology permits up to 5 million sequence reads per lane or a total of 40 millions per an 8-lane flowcell, a density sufficient for quantitative analysis of DNA-protein interactions, gene expression, and other types of applications. As diagrammed in FIG. 9 a biotinylated sequencing primer plus a random 8 mer at the 3′ end (primer A) can be used to reverse transcribe poly(A)+ RNA. The products are random-primed the second time using the second sequencing primer linked to random 8mer (primer B). The products are then biotin-selected to remove free nucleotides as well as excess primer B that remain in the solution. The second primed products are finally released under a high pH condition from the beads, leaving behind the first primed cDNA and unextended primer A. The released products are single-stranded and contain unique sequencing primers at both ends as required for single molecule amplification on the flowcell.

This double-random priming protocol is applicable to any DNA or RNA samples. As illustrated in FIG. 11, the method was used to perform digital analysis of gene expression in LNCaP cells before and after the androgen treatment. The quantitative data are robust, recovering ˜78% of all mRNAs in the RefSeq database and reveal quantitative differences in response to hormone stimulation. These results demonstrate novel methods for performing high throughput sequencing.

Digital analysis of genome rearrangement in prostate cancer: Chromosome translocation events have been detected in both blood disorders and solid tumors, such as breast and prostate cancers, which provide critical insights into cancer etiology. However, it is unknown how many other chromosome translations have taken place and how the human genome is globally rearranged in specific cancer cells. It is hypothesized that multiple genetic lesions, including chromosomal rearrangement, might coordinately contribute to tumor development.

The present methods allow for the global mapping of genetic alterations in model prostate cancer cells.

Cell line choices: three established cell lines comprising prostate cancer cell lines: RWPE1, LNCaP, and DU145 will be analyzed. RWPE1 represents immortalized normal prostate epithelial cells. LNCaP cells are androgen-sensitive whereas DU145 cells have lost androgen responsiveness, which likely represent early and later stage prostate tumors, respectively. In addition, several specific chromosome translocations have been characterized in LNCaP cells, which will provide internal controls for the experiments. DU145 is known to have lost AR and FoxA1 expression, which will serve as an interesting model to study androgen-independent cell growth.

Large-scale pair-end sequencing: The recent discovery of gene fusion events was based on the use of an ingenious bioinformatics approach to identify genes whose expression was dramatically altered in a subset of prostate tumors. However, this method is unlikely to uncover all possible DNA rearrangement events in the genome, which leave open the question of how extensively the genome has been altered. The methods provided herein can be used to perform large-scale sequencing using the double random priming protocol described herein.

Genomic DNA will be isolated from each cell type. The DNA will be shared to smaller pieces by sonication followed by double random priming as illustrated in FIG. 9. The reaction will generate a large number of size-selected amplicoms to load on the sequencer. Using the new module from the Solexa/Illumina sequencer, 35 nt from both ends of individual amplicoms will be sequenced. This will greatly facilitate mapping of sequenced tags back to the human genome to detect rearranged DNA in the chosen prostate cancer cells.

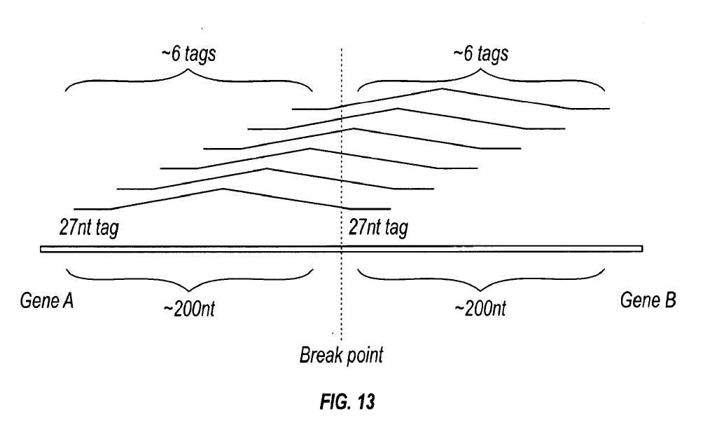

One ug total genomic DNA was routinely used to template random priming and yield final products that are sufficient to generate more than 100 million independent sequence tags. The question here is how many sequencing reads are necessary to detect potential gene fusion products. FIG. 13 uses a hypothetic gene fusion event to aid in the estimation. In this example because of 8 random sequences in both ends of individual amplicoms, 35−=27 nt sequences can be used for genome mapping of the sequenced tags. Under standard conditions, one flowcell can generate 40 million sequence reads, yielding 27×40×10⁶ or about 1×10⁹ nt or ˜0.3× coverage of the human genome. As illustrated in FIG. 15, if it is assumed an average amplicom length of 200 nt, 1× coverage would be possible to generate about 6 sequence tags across a fusion gene event. Based on this estimation, the initial plan is to generate 1× coverage per genome by using 3 flowcells per genetic DNA, although two tags are used that are mapped across a specific break point to infer a potential gene fusion event.

To test this calculation, a pilot experiment is performed using DNA from LNCaP cells, which has been shown to carry several specific gene fusion events. This will allow us to determine the sequence coverage necessary to identify the known gene fusion events. Based on the outcome, the sequence is adjusted for coverage for later large-scale mapping.

Data analysis and validation: a standard pipeline to map sequence tags to the human genome can be used, which involves a systemic blast search for unique match to 27 nt sequence tags from one end (A end) by allowing a maximal of ant mismatches. Next the genomic location is of the sequence tag is determined from the other end (B end). All di-tags that can be uniquely mapped to the human genome into the following categories will be classified: (1) Un-rearranged (i.e. both tags are within 500 nt because amplicoms between 100 to 500 nt are size selected before loading to the flowcell), (2) Rearranged within the same chromosomes (i.e. two ends are more than 500 nt apart, but within the same chromosomes), which may result from deletion, insertion, inversion, other chromosomal abnormalities, (3) Rearranged between chromosomes (i.e. the two ends are mapped to separate chromosomes). Obviously, analysis of the sequencing data will be a huge undertaking, which belongs to a systematic cancer genomics project. Particular attention will be focused on potential chromosome translocation events that are represented by at least two independent amplicoms, which will generate a list of candidate gene fusion events from each cell line to be analyzed by high throughput sequencing.

To validate the candidates, PCR is carried out using specific primers on DNA isolated from individual cell lines. For comparison, DNA from normal diploid human cells purchased from Stratagene will be used. It is expected that most gene fusion events are cell type specific. In other words, the detection of a given hybrid DNA in one cell type or other in others and certainly not in normal human genomic DNA. The PCR products will be sequenced to validate specific gene fusion events. To further validate specific gene fusion events in cells, Q-dot based FISH will be performed using 50 nt primers specific for each end of the fusion gene products, a technique employed in the studies of inter-chromosome interactions. In most cases, one pair of untranslocated foci (separate dots) and one pair of co-localized foci in a given cell type will detect the specific gene fusion event by both sequencing and gene-specific PCR.

Extension of the cell line study to primary prostate tumors: To address the contribution of individual chromosome translocation events to prostate cancer development in patients, DNA from prostate cancer patients will be analyzed. A laser capture microscope will be used to dissect prostate tumors from patients, each of which will be characterized by the Gleason scoring system and roughly divided into several standard clinical stages (i.e. prostatic intraepithelial neoplasia, localized prostate cancer, metastatic prostate cancer, etc.). DNA isolated from the tumor samples will be subject to methods provided herein using specific primers against specific gene fusion products detected in prostate cancer cells.

Elucidation of gene networking in prostate cancer cells: Despite the fact that cancer cells have unstable genome, chromosomal segments are unlikely shuffled in a random fashion. Based on the recent observation of hormone-induced gene networks in MCF7 cells, it is hypothesized that specific and functional inter-chromosome interactions in normal precursor cells may be a prerequisite for aberrant chromosome translocation in cancers. It is proposed to test the hypothesis in the prostate cancer model by developing a general experimental approach to mapping three-dimensional gene networks in the nucleus. The application of the technology to the prostate cancer cell lines will help elucidate the molecular basis for the recently detected chromosome translocation events involving the androgen regulated gene TMPRSS2. The approach should be generally applicable to a wide range of basic and clinical research to understand the architectural basis for regulated gene expression in the nucleus of eukaryotic cells.

The approach will start with the established chromosome conformation capture (3C) assay. The original 3C assay was developed to detect long distance DNA-DNA interactions, which involves in situ restriction digestion of DNA in formaldehyde treated cells, ligation under an extreme dilution condition to promoter ligation of DNAs that are tethered by protein complexes, and PCR analysis of shuffled DNA. Since its introduction, the 3C technology has received wide applications in detecting intra- and inter-chromosomal interactions, and more importantly, various labs have further improved or revised the technology for multiplex applications, enhanced detection by adding an immunoprecipitation step, and unbiased identification of interaction partners by PCR amplification of circularized DNA. It is likely that many other experimental strategies are under development. Here, two complementary global 3C strategies based on high throughput sequencing are provided. A first focus of the technology development effort will be on LNCaP cells. Once the technology becomes matured, application of the technology will test the gene network hypothesis in both normal (e.g. RWPE1) and other prostate cancer (e.g. LNCaP) cell lines.

Development of a 3C strategy: As illustrated in FIG. 12, one of the strategies which is referred to as target-specific global 3C is designed to detect any DNA sequences that are in close contact with a specific DNA element (i.e. gene promoter, enhancer, or other regulatory element) in the nucleus of a given cell type. After the conventional 3C steps, a set of sequence-specific primers will be hybridized to multiple restriction fragments in a target region under investigation. All restriction fragments will be targeted in a chosen region, instead of a particular one closest to the specific DNA element under study, to ensure all possible combinations because different sites may be differentially accessible to surrounding DNA ends. Each of these primers contains a universal sequencing primer (A) at the 5′ end and a target sequence at the 3′ end to anneal to the end of a specific restriction fragment. The 5′ end of the primers will also carry a biotin moiety to allow streptavidin selection. The annealed primers are next extended into ligated DNA segments. After the removal of free nucleotides and primers through a sizing column, the second random priming reaction will use a universal random primer containing the second sequencing primer (B) at the 5′ end and random 8mer at the 3′ end. The rest of steps are similar to the protocol depicted in FIG. 9, including selection on streptavidin beads, release of the second primed DNA from the beads, amplification by PCR, and size selection in an agarose gel. The final size-selected amplicoms are subjected to high throughput sequencing.

Application of the global 3C technology to prostate cancer cells: Some specific chromosome translocation events may reflect normal functional inter-chromosomal interactions in precursor cells. Accordingly, androgen responsive TMPRSS2 gene may engage in inter-chromosomal interactions with other genes including members of the ETS gene family. These interactions may be detectable in normal RWPE1 cells in response to androgen stimulation. Before performing global 3C experiments, first conventional 3C on PWPE1 cells will be carried out before and after the androgen treatment using several primers targeting the TMPRSS2 gene and its partners based on the gene fusion events detected in LNCaP cells. If specific inter-chromosomal interactions could indeed be detected, they will serve as the internal controls for validating the 3C procedure in subsequent global 3C experiments.

The methods provided herein will be used to detect potential gene fusion events in prostate cancer cells. Many of those gene fusion events may be the consequences of inter-chromosomal interactions in precursor cells. Since DU145 cells are representative androgen-insensitive prostate cancer cells, it will be interesting to examine potential gene fusion events, which may shed lights onto the androgen refractory mechanisms. It is proposed that to select a few targets according to newly detected gene fusion events in DU145 cells to perform target-specific global 3C experiments on LNCaP cells before and after androgen treatment. Similar to the validation plan in aim 1, a few examples will be selected for validation by conventional 3C and FISH. Further functional studies may be followed based on newly detected gene fusion events in DU145 cells and the functional relationship with androgen-dependent inter-chromosomal interactions in RWPE1 and LNCaP cells.

Determination of the molecular basis for androgen dependency switch in prostate cancer progression: The present methods can be used to identify potential mechanisms for the development of androgen refractory prostate tumors in humans.

Contribution of specific histone demethylases to androgen independent gene expression and cell proliferation: JMJD3 will be used, which have recently been shown to function as a H2K27 demethylase, and JMJD2C, which has been established as H3K9 demethylase. Because both H2K27 and H3K9 are generally associated with gene silencing, overexpression of these demethylases in prostate cancer cells may be responsible for the activation of critical genes that are sufficient to confer androgen-independent cell proliferation. To directly test this hypothesis, microinjection of the plasmids for the demethylases will be performed in conjunction with a PSA-based reporter (a well known gene that depends on androgen to activate) into LNCaP cells. This will allow us to determine whether overexpression of the demethylases is sufficient to induce PSA expression in the absence of androgen. If this is indeed the case, a stable cell lines will be created to overexpress these demethylases to determine whether the engineered LNCaP cells can growth in the absence of androgen, which can be quantified by Br-dU incorporation. These cells will be further analyzed by using the high throughput sequencing approach to determine how many androgen-responsive genes are constitutively activated in the absence of androgen stimulation.

A number of embodiments of the invention have been described. Nevertheless, it will be understood that various modifications may be made without departing from the spirit and scope of the invention. Accordingly, other embodiments are within the scope of the following claims.

1. A method for determining the sequence of a target polynucleotide, the method comprising:
a) providing a nucleic acid template comprising a target polynucleotide, wherein the template comprises a capture moiety;
b) generating a first hybridization complex by annealing to the target polynucleotide a primer pair comprising:
i) a first oligonucleotide comprising a first portion comprising a target-specific terminal annealing domain partially or completely complementary to a first segment of the target polynucleotide, and a second portion comprising a universal landing site that is complementary to an amplification primer but not complementary to the target polynucleotide;
ii) a second oligonucleotide comprising a first portion comprising a target-specific terminal annealing domain partially or completely complementary to a second segment of the target polynucleotide, and a second portion comprising a universal landing site that is complementary to an amplification primer but not complementary to the target polynucleotide,
wherein the universal landing sites are not the same, and wherein the first oligonucleotide target-specific terminal annealing domain and second oligonucleotide target-specific terminal annealing domain generate a ligatable junction;
c) ligating the ligatable junction to form a ligated probe;
d) separating the ligated probe from the template;
e) hybridizing the ligated probe to a first capture primer comprising a terminal end detachably linked to a solid substrate and an unlinked terminal end, and extending the first capture primer to form a first extended polynucleotide complementary to the ligated probe;
f) removing the ligated probe and annealing the unlinked terminal end of the first extended polynucleotide to a second capture primer comprising a terminal end detachably linked to a solid substrate, and extending the second capture primer to form a second extended polynucleotide complementary to the first extended polynucleotide;
g) optionally repeating part f), wherein a colony of polynucleotides detachably linked to a solid substrate and suitable for sequencing are formed; and
h) determining the sequence of the target polynucleotide.

2. The method of claim 1, wherein the first oligonucleotide or second oligonucleotide, or first oligonucleotide and second oligonucleotide, further comprise a zip code sequence.

3. The method of claim 2, wherein the zip code sequence is juxtaposed between the terminal annealing domain and universal landing site.

4. The method of claim 1, wherein the nucleic acid template is deoxyribonucleic acid (DNA).

5. The method of claim 1, wherein the nucleic acid template is ribonucleic acid (RNA).

6. The method of claim 1, wherein the nucleic acid template is derived from cDNA.

7. The method of claim 1, wherein the target polynucleotide comprises a splice junction.

8. The method of claim 1, wherein the universal landing sites are selected from a T3 and T7 priming site.

9. The method of claim 1, wherein the ligating is performed by a T4 ligase.

10. The method of claim 1, wherein the extending is by PCR.

11. The method of claim 1, wherein the capture moiety is biotin.

12. The method of claim 1, wherein the first oligonucleotide or second oligonucleotide, or first oligonucleotide and second oligonucleotide comprises a detectable label.

13. The method of claim 12, wherein the detectable label is selected from the group consisting of an isotopic label; a magnetic, electrical, or thermal label; an enzymatic label; and a fluorescent or luminescent label.

14. The method of claim 13, wherein the isotopic label comprises a radioactive or heavy isotopes.

15. The method of claim 13, wherein the fluorescent or luminescent label is selected from the group consisting of fluorescent lanthanide complexes, Europium, Terbium, fluorescein, rhodamine, tetramethylrhodamine, eosin, erythrosin, coumarin, methyl-coumarins, quantum dots, pyrene, Malacite green, stilbene, Lucifer Yellow, pyrenyloxytrisulfonic acid, sulforhodamine 101 chloride, Cyanine dyes, alexa dyes, phycoerythin, and bodipy

16. A method for determining the sequence of a target polynucleotide, the method comprising:
a) providing a nucleic acid template comprising a target polynucleotide;
b) annealing a first oligonucleotide to the template, wherein the first oligonucleotide comprises:
i) a first portion comprising a random sequence that partially or completely hybridizes to the target polynucleotide;
ii) a second portion comprising a universal landing site that is complementary to an amplification primer but not complementary to the target polynucleotide; and
iii) a capture moiety,
c) extending the first oligonucleotide in a template directed reaction and isolating a first random priming product;
d) annealing a second oligonucleotide to the first random priming product, wherein the second oligonucleotide comprises:
i) a first portion comprising a random sequence that partially or completely hybridizes to the first random priming product; and
ii) a second portion comprising a universal landing site that is complementary to an amplification primer but not complementary to the first random priming product;
e) extending the second oligonucleotide primer in a template directed reaction and generating a second random priming product annealed to the first random priming product thereby forming a product complex;
f) binding the capture moiety associated with the product complex with a binding partner associated with a solid substrate;
g) separating the second random priming product from the first random priming product;
h) hybridizing the second random priming product to a first capture primer comprising a terminal end detachably linked to a solid substrate, and extending the first capture primer to form a first extended polynucleotide complementary to the ligated probe;
i) removing the ligated probe and annealing the unlinked terminal end of the first extended polynucleotide to a second capture primer comprising a terminal end detachably linked to a solid substrate, and extending the second capture primer to form a second extended polynucleotide complementary to the first extended polynucleotide;
j) optionally repeating part i), wherein a colony of polynucleotides detachably linked to a solid substrate and suitable for sequencing are formed; and
k) determining the sequence of the target polynucleotide.

17. The method of claim 16, wherein the nucleic acid template is deoxyribonucleic acid (DNA).

18. The method of claim 16, wherein the nucleic acid template is ribonucleic acid (RNA).

19. The method of claim 16, wherein the nucleic acid template is derived from cDNA.

20. The method of claim 16, wherein the universal primer comprises an oligo-dT domain for hybridizing to mRNA.

21. The method of claim 16, wherein the template nucleic acid is obtained from a chromatin immunoprecipitation (ChIP) assay.

22. The method of claim 16, wherein the template nucleic acid is obtained from a chromosome conformation capture (3C) assay.
